SEQUENCE LISTING (68 sequences)

SEQ ID NO: 1 - RNA, 20 nt, synthetic construct
  source 1..20: mol_type = other RNA
ggtctgagtagattactcct

SEQ ID NO: 2 - RNA, 20 nt, synthetic construct
  source 1..20: mol_type = other RNA
tgaacaggaaataacctatg

SEQ ID NO: 3 - RNA, 20 nt, synthetic construct
  source 1..20: mol_type = other RNA
tcttatcttaatggcctctg

SEQ ID NO: 4 - DNA, 600 bp, Homo sapiens
  source 1..600: mol_type = genomic DNA
aacttctttattccttagtaatttattgcttctcattgccccagcaataatattttgtcaaatgcagaaaatttatcttttttttttgagtcggagtctcactctgtcgcccaggctggagtgcagtggcacaatctccgctcactgcaacctctgccgcccatgttcaagagattctcctgcctcagcctcccgagtagctaggactacaggcgcctgacatcatgcccgactactttttgtatttttagtagagacggagtttcaccgtgttagccaggatggtctcgatctcctgacctcgtgatcggcatgcctcggcctcccaaagtgctgggattacaggcgtgagccaccgcgcccggcctaaaaatctttttttaaaacaaatattcataagaaacgtgtttaggcttgaagaaaatcagagaaagaactttagattatttaatgcaaaatgagctccaatactcgttctccacctcacccttttaattgcactagggaatcctgtatataaaccatttattaacttcttaactactgttattatagagtacagtccctgacatcacacactgcagagatggataaccaagg

SEQ ID NO: 5 - DNA, 600 bp, Homo sapiens
  source 1..600: mol_type = genomic DNA
agtaatctactcagacctgaatctgcccccaaacccaaagaggcagcaacgaaaacctaaaggcaataaaaactccattttagcaactgaacaggaaataacctatgcggaattaaaccttcaaaaagcttctcaggattttcaagggaatgacaaaacctatcactgcaaaggtaaagcatttaaaagatcctcaatataacagtctaggatgtgcagcttggggtacaggaatgtggggaaagagaagggagtgctcatatatcttctatttgcaaagatcagaattccaagttgagatatgctatttcaatgtaaagtatgaagactgattgaactcattgttgaagtttgtagtctttgtcaaataattcatggagcattatttttcctgaaaattcaatggtatattattctgagaaaaagattacaatgggagatgagggtttggggtccaagtttctctgtatgattcctgtgcattcaggttctcttgtctgtgaatcttctaaacgactgtatccacctctcctttcgcactgttcccatttctctccctgcagatttaccatcagctccagagaagctcattgttgggat

SEQ ID NO: 6 - DNA, 2214 bp, synthetic construct
  source 1..2214: mol_type = other DNA
aggcagcggagagggcagaggaagtcttctaacatgcggtgacgtggaggagaatcccggccctatggagtttgggctgagctggctttttcttgtggctattttaaaaggtgtccagtgctctagagatattttgctgacccaaactccactctccctgcctgtcagtcttggagatcaagcctccatctcttgcagatctagtcagagtcttgtacaccgtaatggaaacacctatttacattggtacctgcagaagccaggccagtctccaaagctcctgattcacaaagtttccaaccgattttctggggtcccagacaggttcagtggcagtggatcagggacagatttcacactcaagatcagcagagtggaggctgaggatctgggagtttatttctgttctcaaagtacacatgttcctccgctcacgttcggtgctgggaccaagctggagctgaaacgggctgatgctgcaccaactgtatccatcttcccaggctcgggcggtggtgggtcgggtggcgaggtgaagcttcagcagtctggacctagcctggtggagcctggcgcttcagtgatgatatcctgcaaggcttctggttcctcattcactggctacaacatgaactgggtgaggcagaacattggaaagagccttgaatggattggagctattgatccttactatggtggaactagctacaaccagaagttcaagggcagggccacattgactgtagacaaatcgtccagcacagcctacatgcacctcaagagcctgacatctgaggactctgcagtctattactgtgtaagcggaatggagtactggggtcaaggaacctcagtcaccgtctcctcagccaaaacgacacccccatcagtctatggaagggtcaccgtctcttcagcggagcccaaatcttgtgacaaaactcacacatgcccaccgtgcccggatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgt

SEQ ID NO: 7 - DNA, 1743 bp, synthetic construct
  source 1..1743: mol_type = other DNA
aggcagcggagagggcagaggaagtcttctaacatgcggtgacgtggaggagaatcccggccctatgcctgaaccctctaagtctgctccagcccctaaaaagggttctaagaaggctatcactaaggcgcagaagaaggatggtaagaagcgtaagcgcagccgcaaggagagctattctatctatgtgtacaaggttctgaagcaggtccaccccgacaccggcatctcatccaaggccatggggatcatgaactccttcgtcaacgacatcttcgagcgcatcgcgggcgaggcttctcgcctggctcactacaataagcgctcgaccatcacctccagggagattcagacggctgtgcgcctgctgctgcctggggagctggctaagcatgctgtgtccgagggcactaaggcagttaccaagtacactagctctaaggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgt

SEQ ID NO: 8 - DNA, 1098 bp, synthetic construct
  source 1..1098: mol_type = other DNA
aagcttatggtggtcatggcgccccgaaccctcttcctgctgctctcgggggccctgaccctgaccgagacctgggcgggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctactacaatcagagcgaggccgggtctcacaccctgcagtggatgcatggctgcgagctggggcccgacaggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaagaattc

SEQ ID NO: 9 - DNA, 22 bp, synthetic construct
  source 1..22: mol_type = other DNA
cggagtctcactctgtcgccca

SEQ ID NO: 10 - DNA, 23 bp, synthetic construct
  source 1..23: mol_type = other DNA
tggaccccaaaccctcatctccc

SEQ ID NO: 11 - DNA, 25 bp, synthetic construct
  source 1..25: mol_type = other DNA
tgtgcagaccacatagtcttaacca

SEQ ID NO: 12 - DNA, 22 bp, synthetic construct
  source 1..22: mol_type = other DNA
aggaaccagaagccttgcagga

SEQ ID NO: 13 - DNA, 25 bp, synthetic construct
  source 1..25: mol_type = other DNA
tgtgcagaccacatagtcttaacca

SEQ ID NO: 14 - DNA, 22 bp, synthetic construct
  source 1..22: mol_type = other DNA
tgccttagtgccctcggacaca

SEQ ID NO: 15 - DNA, 24 bp, synthetic construct
  source 1..24: mol_type = other DNA
agatttgggggaagcactgaagtc

SEQ ID NO: 16 - DNA, 22 bp, synthetic construct
  source 1..22: mol_type = other DNA
gggggtgtcgttttggctgagg

SEQ ID NO: 17 - DNA, 25 bp, synthetic construct
  source 1..25: mol_type = other DNA
ctggcttttgtgttacctgcttgac

SEQ ID NO: 18 - DNA, 22 bp, synthetic construct
  source 1..22: mol_type = other DNA
gggggtgtcgttttggctgagg

SEQ ID NO: 19 - DNA, 25 bp, synthetic construct
  source 1..25: mol_type = other DNA
tatgcagggagcagggaggatatag

SEQ ID NO: 20 - DNA, 22 bp, synthetic construct
  source 1..22: mol_type = other DNA
gggggtgtcgttttggctgagg

SEQ ID NO: 21 - DNA, 22 bp, synthetic construct
  source 1..22: mol_type = other DNA
tgggaggctgaggcaggagaac

SEQ ID NO: 22 - DNA, 22 bp, synthetic construct
  source 1..22: mol_type = other DNA
acagttggtgcagcatcagccc

SEQ ID NO: 23 - DNA, 24 bp, synthetic construct
  source 1..24: mol_type = other DNA
aatattggcaggaaggcaggacag

SEQ ID NO: 24 - DNA, 22 bp, synthetic construct
  source 1..22: mol_type = other DNA
acagttggtgcagcatcagccc

SEQ ID NO: 25 - DNA, 19 bp, synthetic construct
  source 1..19: mol_type = other DNA
caaagggaccctgcccacc

SEQ ID NO: 26 - DNA, 22 bp, synthetic construct
  source 1..22: mol_type = other DNA
gggggtgtcgttttggctgagg

SEQ ID NO: 27 - DNA, 17 bp, synthetic construct
  source 1..17: mol_type = other DNA
cacgggcaatccccgca

SEQ ID NO: 28 - DNA, 22 bp, synthetic construct
  source 1..22: mol_type = other DNA
gggggtgtcgttttggctgagg

SEQ ID NO: 29 - DNA, 23 bp, synthetic construct
  source 1..23: mol_type = other DNA
tggtcatgcttggaagacccttg

SEQ ID NO: 30 - DNA, 22 bp, synthetic construct
  source 1..22: mol_type = other DNA
gggggtgtcgttttggctgagg

SEQ ID NO: 31 - DNA, 23 bp, synthetic construct
  source 1..23: mol_type = other DNA
tccaagactgcttttcctgtggg

SEQ ID NO: 32 - DNA, 22 bp, synthetic construct
  source 1..22: mol_type = other DNA
gggggtgtcgttttggctgagg

SEQ ID NO: 33 - DNA, 24 bp, synthetic construct
  source 1..24: mol_type = other DNA
gcgcagaagagtgtaagaaggtca

SEQ ID NO: 34 - DNA, 22 bp, synthetic construct
  source 1..22: mol_type = other DNA
gggggtgtcgttttggctgagg

SEQ ID NO: 35 - DNA, 25 bp, synthetic construct
  source 1..25: mol_type = other DNA
ctccatggcagcatttctcatttgg

SEQ ID NO: 36 - DNA, 22 bp, synthetic construct
  source 1..22: mol_type = other DNA
gggggtgtcgttttggctgagg

SEQ ID NO: 37 - DNA, 22 bp, synthetic construct
  source 1..22: mol_type = other DNA
caccccatgcacacacctccac

SEQ ID NO: 38 - DNA, 22 bp, synthetic construct
  source 1..22: mol_type = other DNA
gggggtgtcgttttggctgagg

SEQ ID NO: 39 - DNA, 22 bp, synthetic construct
  source 1..22: mol_type = other DNA
gggggtgtcgttttggctgagg

SEQ ID NO: 40 - DNA, 25 bp, synthetic construct
  source 1..25: mol_type = other DNA
tgccaagacaagtcaggtttgatga

SEQ ID NO: 41 - DNA, 22 bp, synthetic construct
  source 1..22: mol_type = other DNA
gggggtgtcgttttggctgagg

SEQ ID NO: 42 - DNA, 20 bp, synthetic construct
  source 1..20: mol_type = other DNA
gccccattctccgtacctgc

SEQ ID NO: 43 - DNA, 22 bp, synthetic construct
  source 1..22: mol_type = other DNA
gggggtgtcgttttggctgagg

SEQ ID NO: 44 - DNA, 23 bp, synthetic construct
  source 1..23: mol_type = other DNA
tagtgcttgacccatgcagaacc

SEQ ID NO: 45 - DNA, 22 bp, synthetic construct
  source 1..22: mol_type = other DNA
gggggtgtcgttttggctgagg

SEQ ID NO: 46 - DNA, 22 bp, synthetic construct
  source 1..22: mol_type = other DNA
ctggctgaagtgggcgtccttg

SEQ ID NO: 47 - DNA, 22 bp, synthetic construct
  source 1..22: mol_type = other DNA
gggggtgtcgttttggctgagg

SEQ ID NO: 48 - DNA, 22 bp, synthetic construct
  source 1..22: mol_type = other DNA
gaattctgtctggcaggtcccc

SEQ ID NO: 49 - DNA, 22 bp, synthetic construct
  source 1..22: mol_type = other DNA
gggggtgtcgttttggctgagg

SEQ ID NO: 50 - DNA, 24 bp, synthetic construct
  source 1..24: mol_type = other DNA
ttcttcctctctgtcccctgagtc

SEQ ID NO: 51 - DNA, 22 bp, synthetic construct
  source 1..22: mol_type = other DNA
gggggtgtcgttttggctgagg

SEQ ID NO: 52 - DNA, 20 bp, synthetic construct
  source 1..20: mol_type = other DNA
aatcaggggctaccgttgcg

SEQ ID NO: 53 - DNA, 22 bp, synthetic construct
  source 1..22: mol_type = other DNA
gggggtgtcgttttggctgagg

SEQ ID NO: 54 - DNA, 24 bp, synthetic construct
  source 1..24: mol_type = other DNA
tgcagaacccatgaccaatctctg

SEQ ID NO: 55 - DNA, 22 bp, synthetic construct
  source 1..22: mol_type = other DNA
gggggtgtcgttttggctgagg

SEQ ID NO: 56 - DNA, 21 bp, synthetic construct
  source 1..21: mol_type = other DNA
aacagcccatctcaaggcacc

SEQ ID NO: 57 - DNA, 22 bp, synthetic construct
  source 1..22: mol_type = other DNA
gggggtgtcgttttggctgagg

SEQ ID NO: 58 - DNA, 23 bp, synthetic construct
  source 1..23: mol_type = other DNA
gcctcttcccgtttgaggtttct

SEQ ID NO: 59 - DNA, 22 bp, synthetic construct
  source 1..22: mol_type = other DNA
gggggtgtcgttttggctgagg

SEQ ID NO: 60 - DNA, 24 bp, synthetic construct
  source 1..24: mol_type = other DNA
ccctcccctctgggatagagtttt

SEQ ID NO: 61 - DNA, 22 bp, synthetic construct
  source 1..22: mol_type = other DNA
gggggtgtcgttttggctgagg

SEQ ID NO: 62 - DNA, 23 bp, synthetic construct
  source 1..23: mol_type = other DNA
ctgacatttcaccaagggagcca

SEQ ID NO: 63 - DNA, 20 bp, synthetic construct
  source 1..20: mol_type = other DNA
tccctgacatcacacactgc

SEQ ID NO: 64 - DNA, 20 bp, synthetic construct
  source 1..20: mol_type = other DNA
tgcctttaggttttcgttgc

SEQ ID NO: 65 - protein, 1368 aa, Streptococcus pyogenes [sequence omitted]

SEQ ID NO: 66 - protein, 1409 aa, Streptococcus thermophilus
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 67 - protein, 1082 aa, Neisseria meningitidis
MAAFKPNPINYILGLDIGIASVGWAMVEIDEEENPIRLIDLGVRVFERAEVPKTGDSLAMVRRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHISGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 68 - protein, 1395 aa, Treponema denticola
MKKEIKDYFLGLDVGTGSVGWAVTDTDYKLLKANRKDLWGMRCFETAETAEVRRLHRGARRRIERRKKRIKLLQELFSQEIAKTDEGFFQRMKESPFYAEDKTILQENTLFNDKDFADKTYHKAYPTINHLIKAWIENKVKPDPRLLYLACHNIIKKRGHFLFEGDFDSENQFDTSIQALFEYLREDMEVDIDADSQKVKEILKDSSLKNSEKQSRLNKILGLKPSDKQKKAITNLISGNKINFADLYDNPDLKDAEKNSISFSKDDFDALSDDLASILGDSFELLLKAKAVYNCSVLSKVIGDEQYLSFAKVKIYEKHKTDLTKLKNVIKKHFPKDYKKVFGYNKNEKNNNNYSGYVGVCKTKSKKLIINNSVNQEDFYKFLKTILSAKSEIKEVNDILTEIETGTFLPKQISKSNAEIPYQLRKMELEKILSNAEKHFSFLKQKDEKGLSHSEKIIMLLTFKIPYYIGPINDNHKKFFPDRCWVVKKEKSPSGKTTPWNFFDHIDKEKTAEAFITSRTNFCTYLVGESVLPKSSLLYSEYTVLNEINNLQIIIDGKNICDIKLKQKIYEDLFKKYKKITQKQISTFIKHEGICNKTDEVIILGIDKECTSSLKSYIELKNIFGKQVDEISTKNMLEEIIRWATIYDEGEGKTILKTKIKAEYGKYCSDEQIKKILNLKFSGWGRLSRKFLETVTSEMPGFSEPVNIITAMRETQNNLMELLSSEFTFTENIKKINSGFEDAEKQFSYDGLVKPLFLSPSVKKMLWQTLKLVKEISHITQAPPKKIFIEMAKGAELEPARTKTRLKILQDLYNNCKNDADAFSSEIKDLSGKIENEDNLRLRSDKLYLYYTQLGKCMYCGKPIEIGHVFDTSNYDIDHIYPQSKIKDDSISNRVLVCSSCNKNKEDKYPLKSEIQSKQRGFWNFLQRNNFISLEKLNRLTRATPISDDETAKFIARQLVETRQATKVAAKVLEKMFPETKIVYSKAETVSMFRNKFDIVKCREINDFHHAHDAYLNIVVGNVYNTKFTNNPWNFIKEKRDNPKIADTYNYYKVFDYDVKRNNITAWEKGKTIITVKDMLKRNTPIYTRQAACKKGELFNQTIMKKGLGQHPLKKEGPFSNISKYGGYNKVSAAYYTLIEYEEKGNKIRSLETIPLYLVKDIQKDQDVLKSYLTDLLGKKEFKILVPKIKINSLLKINGFPCHITGKTNDSFLLRPAVQFCCSNNEVLYFKKIIRFSEIRSQREKIGKTISPYEDLSFRSYIKENLWKKTKNDEIGEKEFYDLLQKKNLEIYDMLLTKHKDTIYKKRPNSATIDILVKGKEKFKSLIIENQFEVILEILKLFSATRNVSDLQHIGGSKYSGVAKIGNKISSLDNCILIYQSITGIFEKRIDLLKV

(19) United States
(12) Patent Application Publication
(10) Pub. No.: US 2025/0255958 A1
(43) Pub. Date: Aug. 14, 2025

(54) VIRUS-FREE CRISPR CAR NATURAL KILLER CELLS

(71) Applicant: Wisconsin Alumni Research Foundation, Madison, WI (US)

(72) Inventors: Krishanu Saha, Middleton, WI (US); Keerthana Shankar, Madison, WI (US)

(73) Assignee: Wisconsin Alumni Research Foundation, Madison, WI (US)

(21) Appl. No.: 19/049,850

(22) Filed: Feb. 10, 2025

Related U.S. Application Data
(60) Provisional application No. 63/552,269, filed on Feb. 12, 2024.

(51) Int. Cl. A61K 40/15; A61K 40/31; A61K 40/42; C07K 16/30; C12N 5/0783; C12N 9/22; C12N 15/11; C12N 15/90

(52) U.S. Cl. CPC A61K 40/15; A61K 40/31; A61K 40/4258; C07K 16/3084; C12N 5/0646; C12N 9/22; C12N 15/11; C12N 15/907; C07K 2317/622; C07K 2319/03; C12N 2310/20; C12N 2510/00

(57) ABSTRACT

Described herein is a genetically modified natural killer (NK) cell including a knock-out of at least a portion of an endogenous gene encoding an NKG2A receptor which inactivates the NKG2A receptor, and a knock-in of a transgene for a chimeric antigen receptor (CAR) into the site of the NKG2A knock-out, wherein the CAR includes an extracellular domain linked to an intracellular domain through a first transmembrane domain, wherein the first extracellular domain comprises an antigen recognition domain. Also described are methods of making and methods of using the genetically modified NK cells.

### CROSS-REFERENCE TO RELATED APPLICATIONS

This application claims priority to U.S. Provisional Application 63/552,269 filed on Feb. 12, 2024, which is incorporated herein by reference in its entirety.

### STATEMENT REGARDING FEDERALLY SPONSORED RESEARCH & DEVELOPMENT

This invention was made with government support under GM119644 awarded by the National Institutes of Health and under EEC1648035 awarded by the National Science Foundation. The government has certain rights in the invention.

### SEQUENCE LISTING

The Instant Application contains a Sequence Listing which has been submitted electronically in XML format and is hereby incorporated by reference in its entirety. Said XML copy, created on Jan. 28, 2025, is named “SEQ_LIST-107668311-P240105US02.xml” and is 72,321 bytes in size. The Sequence Listing does not go beyond the disclosure in the application as filed.

### FIELD OF THE DISCLOSURE

The present disclosure is related to genetically modified natural killer (NK) cells wherein least a portion of an endogenous gene encoding an NKG2A receptor is knocked-out and replaced with a transgene for a chimeric antigen receptor (CAR). The genetically modified NK cells are particularly useful for treating cancer, including solid tumors.

### BACKGROUND

Chimeric antigen receptor (CAR) therapies, which involve genetically engineering patient immune cells to recognize and eliminate cancer antigens, have revolutionized cancer treatment. Despite tremendous success in the clinic, CAR T cells have been shown to induce cytokine release syndrome (CRS), immune effector cell-associated neurotoxicity syndrome (ICANS), and Graft vs Host Disease (GvHD) upon infusion. Moreover, CAR T cell therapies are also restricted to autologous settings due to expression of the T cell receptor (TCR). As an alternative approach, natural killer (NK) cells provide an excellent platform for cell therapies due to their intrinsic ability to eliminate malignancies in an HLA-agnostic manner. CAR NK cells have displayed outstanding results in the clinic for treating hematological malignancies, with 30% of patients achieving a complete response with no instances of CRS, ICANS, or GvHD.

Current CAR NK cells, however, fail to produce comparable results in solid tumors due to the greater upregulation of NK inhibitory ligands and checkpoints by the tumor. What is needed are new methods to prepare CAR NK cells, particularly CAR NK cells that are effective against solid tumors.

### BRIEF SUMMARY

In an aspect, a genetically modified natural killer (NK) cell comprises a knock-out of at least a portion of an endogenous gene encoding an NKG2A receptor which inactivates the NKG2A receptor, and a knock-in of a transgene for a chimeric antigen receptor (CAR) into the site of the NKG2A knock-out, wherein the CAR comprises an extracellular domain linked to an intracellular domain through a first transmembrane domain, wherein the first extracellular domain comprises an antigen recognition domain that binds a tumor-specific antigen.

In another aspect, an ex vivo, non-viral method of site-specifically inserting a transgene containing a chimeric antigen receptor (CAR) gene into an endogenous NK cell gene, to provide genetically modified NK cells, wherein the endogenous NK cell gene encodes an NKG2A receptor comprises providing a non-viral homology-directed repair (HDR) template comprising the transgene flanked by homology arms that are complementary to sequences on both sides of a cleavage site in the endogenous NK cell gene, wherein the CAR comprises an extracellular domain linked to an intracellular domain through a first transmembrane domain, wherein the first extracellular domain comprises an antigen recognition domain that binds a tumor-specific antigen, and wherein cleavage at the cleavage site inactivates the NKG2A receptor; and introducing into a population of unmodified NK cells a Cas9 ribonucleoprotein (RNP) complex and the double-stranded HDR template, to provide the genetically modified NK cells. The Cas9 RNP comprises a Cas9 protein and a guide RNA that directs double stranded DNA cleavage of the cleavage site in the endogenous NK gene. In the method, after introducing the Cas9 RNP and the HDR template, the endogenous NK cell gene is knocked-out and the transgene is specifically knocked-in to the knock-out site, to provide the genetically modified NK cells.

### BRIEF DESCRIPTION OF THE DRAWINGS

FIGS. 1A-I show Cas9 ribonucleoprotein-based strategy yields high KLRC1 knock-out efficiency. (1A) Schematic showing the interaction between NK cells and the tumor. The HLA-E ligand is often upregulated on the surface of tumors and inhibits NK effector functions by binding the inhibitory NKG2A receptor. (1B) Schematic depicting knock-out strategy. sgRNA1 (underlined), in complex with the SpCas9 editor, targets the third exon of the KLRC1 gene. The locations of the three gRNA sequences are indicated by black arrows in sequential order. (1C) Percent of indel formations for each of the three sgRNAs in NK-92 cells at the KLRC1 gene. Data is shown for three replicates for one donor (n=1). (1D) Quantification of the top five allelic editing outcomes of gRNA1 as analyzed by CRISPResso2. (1E) Primary PB-NK cells were nucleofected with RNP complexes targeting KLRCL. Percent of NKG2A⁻ cells is shown in comparison to the UTF population, as measured by flow cytometry. Data is shown as the average of two replicates across four donors (n=4). (1F) MFI of the NKG2A receptor on the cell surface of KLRC1-KO and UTF cells. Data is shown as the average of two replicates across four donors (n=4). (1G and 1H) Cell viability and proliferation of KLRC1-KO and UTF cells after nucleofection. Data is shown as the average of two replicates across two donors (n=2). (1I) Calculated fold expansion of KLRC1-KO and UTF cells eight days after nucleofection. A two-tailed paired t test and one-way ANOVA followed by Tukey's multiple comparisons test were used to test for statistical significance. *, p≤0.05; ***, p≤0.001; ns, p≥0.05. PB, peripheral blood; RNP, ribonucleoprotein; UTF, untransfected; MFI, mean fluorescence intensity.

FIGS. 2A-H show non-viral transgene knock-in into primary NK cells. (2A) Schematic depicting virus-free insertion of the mCherry fluorescence gene at the third exon of the KLRC1 locus. (2B and 2C) NK cells were nucleofected on day 4 of expansion. Percentage of mCherry⁺ cells and NKG2A⁻ cells is shown, as measured by flow cytometry, one week after nucleofection. Data is shown for six replicates for one donor (n=1). (2D) Cell proliferation of KLRC1-No CAR cells from B. (2E) NK cells were incubated with varying concentrations of M3814 for 24 hours after nucleofection. The percent of mCherry⁺ cells was measured by flow cytometry one week after nucleofection. Data is shown for two replicates for one donor (n=1). (2F) Expansion of mCherry⁺ cells after a 24-hour incubation with M3814. (2G) NK cells were nucleofected on day 4 using different pulse programs, followed by M3814 treatment. Data is shown for two replicates for one donor (n=1). (2H) Expansion of NK cells following nucleofection with different pulse programs. Statistical significance was calculated using a two-tailed unpaired t test (2B and 2C) and by ordinary one-way ANOVA. Dunnett's multiple comparison test was used as the post-test (2E and 2G). *, p≤0.05; **, p≤0.01; ***, p≤0.001; ****, p≤0.0001. ANOVA, analysis of variance.

FIGS. 3A-K show characterization of non-viral KLRC1-CAR NK cells. (3A) Proposed manufacturing timeline for KLRC1-CAR NK cells. NK cells are isolated from whole blood of healthy donors using negative selection and activated with irradiated K562-mb15-41BBL cells (1 NK: 2 K562) along with IL-2 and IL-15. On day 4, NK cells are nucleofected with Cas9 RNP and dsDNA HDRT using program EH-100, followed by a 24-hour treatment of 0.5 μM M3814. Nucleofected NK cells are recovered with addition of irradiated K562-mb15-41BBL cells (1:2), supplemented with IL-2 and IL-15. KLRC1-CAR cells are expanded ex vivo for downstream analysis and cell banking. The calculated cell yield reflects the estimated number of KLRC1-CAR⁺ NK cells derived from 60 mL of blood on Day 13 of expansion. (3B) Schematic depicting virus-free insertion of an anti-GD2 CAR at the third exon of the KLRC1 locus. (3C) Representative histogram plots showing knockout of NKG2A and knock-in of CAR or mCherry transgenes one week after nucleofection. X-axis describes protein expression levels. Y-axis describes frequency after normalization to the mode. UTF samples are shown as striped histograms in all plots. The percent shown for each gate represents protein levels in the KLRC1-KO (left), KLRC1-No CAR (middle), and KLRC1-CAR (right) edited samples. (3D and 3E) CAR and mCherry knock-in efficiencies, and MFI levels are shown. Data is shown as an average of two replicates across five donors (n=5). (3F) The percent of CD56⁺CD16⁺ and CD56⁺CD16⁻ cells in each cell population is shown. Data is shown as an average of two replicates across two donors (n=2). (G) The percent of NKG2C+ cells in each population is shown. Data is shown as an average of two replicates for each of the four donors (n=4). (3H and 3I) Cell viability and cell expansion of KLRC1-CAR, KLRC1-No CAR, and KLRC1-KO NK cells after nucleofection are shown. During expansion, multiple nucleofection reactions were pooled to form one sample per group. Data is normalized to the number of reactions for each of the three donors (n=3). (3J) Calculated fold change expansion of KLRC1-CAR, KLRC1-No CAR, and KLRC1-KO NK cells from H is shown in comparison to donor-matched UTF cells, with each donor indicated by a different shape (circle, triangle, diamond). (3K) In-out PCR showing on-target integration of the CAR and mCherry transgenes across two donors. The UTF samples were amplified using the CAR (UTF (C)) and mCherry (UTF (m)) primer pairs, as controls. NTC refers to the PCR reaction performed without the genomic DNA. The primers are indicated by the black arrows on each template in the schematics of FIGS. 2A and 3B. Statistical significance was calculated using an ordinary one-way or two-way ANOVA. Post-tests were performed using the uncorrected Fisher's LSD (D), Sidak's multiple comparisons test (E), and Dunnett's multiple comparisons test (G and J). *, p≤0.05; ****, p≤0.0001; ns, p≥0.05. RNP, ribonucleoprotein; dsDNA, double stranded DNA; HDRT, homology directed repair template; CAR, chimeric antigen receptor; MFI, mean fluorescence intensity; UTF, untransfected; PCR, polymerase chain reaction; NTC, non-template control; ANOVA, analysis of variance.

FIGS. 4A-G show genome-wide off-target analysis. (4A) Nominated off-target sites detected by CHANGE-Seq are organized by number of read counts. The on-target site is the second line on the list, indicated by a black arrow. Base pair mismatches between the sgRNA and the off-target sites are indicated by the nucleotide (n=1). (4B) Editing across the top 12 nominated off-target sites was assayed via amplicon using the rhAmpSeq system. Data is shown as mean (SD) across three donors, with two replicates per donor (n=3). (4C) Schematic depicting locations of the forward and reverse primers for in-out PCRs for the CAR gene at an off-target site. The left schematic represents CAR integration in the 5′-3′ orientation, while the right represents integration in the 3′-5′ orientation, with the primers indicated by black arrows. (4D) In-out PCRs were performed on genomic DNA extracted from KLRC1-CAR NK cells to assess for off-target integration in the forward (left) and reverse (right) orientations. The on-target CAR integration is indicated by the black arrow. (4E) Schematic of the methods used to analyze long-read WGS data. Following WGS, reads containing the CAR transgene were aligned to the human genome and filtered by map quality and overlap window. (4F) Alignment statistics for each of the 16 reads containing the CAR transgene. The reads marked by an asterisk indicate contiguous mapping of the CAR construct several thousand base pairs upstream of the LHA and downstream of the RHA. (4G) The percentage of all 16 reads aligning to each of the identified chromosomal regions. A two-way ANOVA was used to calculate statistical significance, with an Uncorrected Fisher's LSD post-test (4B). PCR, polymerase chain reaction; CAR, chimeric antigen receptor.

FIGS. 5A-F show KLRC1-CAR cells display improved cytotoxicity against GD2+ melanoma cells in vitro. (5A) Flow cytometry histogram plot depicting GD2 expression on the surface of M21 melanoma cells. (5B) Schematic depicting the in vitro potency assay used to assess NK cell cytotoxicity. (5C) Following FACS, NK cells were co-cultured with M21 cells at a 5:1 E:T ratio for 24 hours. For ADCC conditions, post-FACS NK cells were co-cultured with M21 cells in the presence of 500 ng/mL hu14.18K322A anti-GD2 monoclonal antibody. The non-ADCC conditions are indicated with a single NK cell icon above the graph, while the ADCC conditions are indicated by cell and antibody icons. Cytotoxicity was measured by quantifying secreted LDH with the CyQuant LDH Cytotoxicity Assay kit. Data is shown as the mean (SD) across three donors, with 5-6 replicates per donor (n=3). (5D) Cytotoxicity was measured by the same LDH release assay as in C, but for CAR⁺ and CAR⁻ fractions within the same sample. Data is shown as the mean (SD) across three donors, with 5-6 replicates per donor (n=3). (5E and 5F) NK cell secreted IFNγ was measured using an ELISA on media supernatant taken from the 24-hour co-culture with M21 cells for the target cells only (5E) and the ADCC (5F) conditions. Data is shown as mean (SD) for three donors with 3 replicates (n=3) for the target cells only (5E) and two donors with 3 replicates (n=2) for the ADCC (5F) conditions. Statistical significance was calculated using an ordinary two-way ANOVA, with the Dunnett's multiple comparison test (5C), and the Sidak's multiple comparison post-test (5D), and a one-way ANOVA with the Tukey's multiple comparison post-test (E and F). *, P≤0.05; **, P≤0.01; ***, P≤0.001; ****, P≤0.0001; ns, P≥0.05. LDH, lactate dehydrogenase; ADCC, antibody dependent cellular cytotoxicity; CAR, chimeric antigen receptor; ANOVA, analysis of variance.

FIGS. 6A-E show overcoming the NKG2A-HLA-E based checkpoint. (6A) Flow cytometry histogram plot showing HLA-E expression on wild-type M21 cells, HLA-E transduced M21 cells, and HLA-E transduced M21 cells treated with 100 ng/mL of IFNγ for 24 hours. (6B) Schematic depicting the in vitro potency assay used to assess NK cell cytotoxicity against the M21-HLA-E cells. (6C) After FACS, NK cells were co-cultured with M21-HLA-E cells at 0.1:1, 0.5:1, and 1:1 effector-target ratios for 24 hours. Cytotoxicity was measured by quantifying secreted LDH with the CyQuant LDH Cytotoxicity Assay kit. Data is shown as the mean (SD) of five replicates for four donors (n=4). (6D) Potency data is shown for one representative donor from (C) at 0.1:1, 0.5:1, and 1:1 E:T ratios. Data is shown as the mean (SD) of five replicates. (6E) KLRC1-CAR NK samples were sorted into CAR⁻ and CAR⁺ fractions and were cultured with M21-HLA-E cells under the same conditions as in C and D. The secreted LDH was used to quantify NK cytotoxicity after 24 hours. Data is shown as the mean (SD) of five replicates for four donors (n=4). Statistical significance was calculated using an ordinary two-way ANOVA, with the Dunnett's multiple comparison test (C and D), and a two-way ANOVA with the Sidak's multiple comparison post-test (E). *, P≤0.05; **, P≤0.01; ***, P≤0.001; ****, P≤0.0001; ns, P≥0.05. LDH, lactate dehydrogenase; ADCC, antibody dependent cellular cytotoxicity; CAR, chimeric antigen receptor; ANOVA, analysis of variance.

FIGS. 7A-H show KLRC1-CAR NK cells demonstrate improved killing of HLA-E/GD2+ target cells at the single-cell level. (7A) Schematic describing the co-culture setup for time-lapse imaging. (7B and 7C) Representative images of timelapse microscopy of UTF (B), and KLRC1-CAR NK (7C) NK cells over 16 hours. Scale bar denotes 200 m length. (7D) The change in fluorescence intensity over 16 hours for KLRC1-CAR NK and UTF NK cells. Donor 1 is represented with a solid line and donor 2 by the dashed line. Data is shown as the mean (SD) of three replicates for each donor (n=2). (7E) The difference in fluorescence intensity between 0 and 16 hours was quantified for both effector groups. Data is shown as the mean (SD) of three replicates for two donors (n=2). (7F) The percentage of NK cells within each effector group that killed at least one M21-HLA-E target cell. Data is shown as the mean of three replicates for two donors (n=2). (7G) The number of M21-HLA-E target cells killed by an individual NK cell. Data is shown as the mean (SD) of three replicates for two donors (n=2). (7H) Representative image of UTF NK cells and KLRC1-CAR NK cells lysing M21-HLA-E cells. KLRC1-CAR NK cells have the ability to kill multiple M21-HLA-E target cells simultaneously. Statistical significance was calculated using a two-tailed t-test (7E, 7G, and 7H), and ordinary two-way ANOVA with an Uncorrected Fisher's LSD post-test (7F).

The above-described and other features will be appreciated and understood by those skilled in the art from the following detailed description, drawings, and appended claims.

### DETAILED DESCRIPTION

As explained in the background, while CAR NK cells have been successful in treating hematological malignancies, they have failed to produce comparable results in solid tumors due to the greater upregulation of NK inhibitory ligands and checkpoints by the tumor. One of these NK inhibitory ligands, HLA-E, is upregulated across several tumors and is known to limit NK effector function by ligating the inhibitory NKG2A receptor. Blockade of NKG2A by monoclonal antibodies, or ablation of the receptor through genomic engineering has been shown to improve NK effector function in vivo as well as in clinical trials. Combinatorial therapies utilizing the blockade of NKG2A and genomic modifications such as CARs present a large potential for solid tumor immunotherapies.

Another issue is that the innate ontogeny of NK cells confers a high sensitivity to exogenous DNA, making genomic engineering of NK cells profoundly challenging, often resulting in low efficiencies (<1-20%) and poor cell viability. Current engineering techniques rely on viral vectors, which have random integration profiles with insertional mutagenesis risks. Electroporation-based methods that forego a viral approach can deliver mRNA payloads efficiently, but result in a transient gene expression of the mRNA. Alternatively, delivery of transposable vector elements into NK cells results in multiple potential integration sites, also with insertional mutagenesis risks. None of these approaches allow for a precise genome edit in which a specific gene can be targeted for knock-out or transgene insertion. Non-viral genome editing of NK cells with CRISPR genome editors has been achieved, but with low efficiency for large transgene insertion. Virus-free knock-in efficiencies range from 3-17% for fluorescent proteins like GFP (1.5 kb insertion) and mCherry (2.1 kb insertion) transgenes, and less than 10% for a CAR (1.4 kb insertion).

Described herein are genetically modified NK cells and methods using nonviral CRISPR-Cas9 ribonucleoproteins (RNP) to generate potent CAR NK cells against solid tumors. The genome editing strategy relies on the endogenous homology-directed repair (HDR) machinery within NK cells to ensure precise transgene knock-in of long transgenes, including a CAR. Several parameters influencing editing efficiencies have been identified to ensure efficient knock-out of the KLRC1 gene, encoding the inhibitory NKG2A receptor, and knock-in of an anti-GD2 CAR transgene at this locus. Specifically, the GD2 antigen was chosen as a target due to its ubiquitous overexpression in several solid tumors, which makes the CAR NK cells a viable candidate for treating various malignancies, including melanomas, neuroblastomas, and osteosarcomas. KLRC1-CAR NK cells are highly viable and proliferative, with minimal off-target editing. In a GD2⁺ in vitro melanoma model, the KLRC1-CAR NK cells display improved cytotoxicity and secretion of IFNγ when compared to their unedited counterparts. Due to knock-out of NKG2A, the KLRC1-CAR NK cells are also able to overcome HLA-E mediated NK cell inhibition. The streamlined virus-free engineering platform described herein has high potential to generate allogeneic NK cell immunotherapies.

### Genetically Modified NK Cells

In an aspect, a genetically modified natural killer (NK) cell comprises a knock-out of at least a portion of gene encoding an endogenous NKG2A receptor which inactivates the NKG2A receptor, and a knock-in of an expression construct for a chimeric antigen receptor (CAR) into the site of the NKG2A knockout, wherein the CAR comprises a first extracellular domain linked to a first intracellular domain through a first transmembrane domain, wherein the first extracellular domain comprises an antigen recognition domain, e.g., an antigen recognition domain that binds a tumor-specific antigen.

Natural Killer (NK) cells are cytotoxic, HLA-agnostic lymphocytes that have an innate ability to eliminate tumors. Their HLA-agnostic nature makes them an excellent candidate for off-the-shelf therapies, due to the decreased likelihood of graft vs. host disease (GvHD). Despite their impressive abilities, NK cells are often faced with inhibitory factors or immunosuppressive proteins within the tumor microenvironment. One of those inhibitory factors is the HLA-E ligand that is often upregulated on tumors and that binds the inhibitory NKG2A receptor on NK cells. Described herein is a knock-out of the NKG2A receptor and replacement with a chimeric antigen receptor that recognizes an antigen on the tumor.

The unmodified NK cells can be obtained from a subject in need of therapy or suffering from a disease associated with reduced immune cell activity. Thus, the cells will be autologous to the subject in need of therapy. Alternatively, the unmodified NK cells can be obtained from a donor such as an allogenic healthy donor. The unmodified NK cells can be harvested from PB, cord blood, bone marrow, spleen, or any other organ/tissue in which immune cells reside in said subject or donor. The unmodified NK cells can be isolated from a pool of subjects and/or donors, such as from pooled cord blood.

### Chimeric Antigen Receptors

A CAR comprises a first extracellular domain linked to a first intracellular domain through a first transmembrane domain, wherein the first extracellular domain comprises an antigen recognition domain.

An antigen-specific extracellular domain specifically binds an antigen when, for example, it binds the antigen with an affinity constant or affinity of interaction (KD) between about 0.1 pM to about 10 μM, specifically about 0.1 pM to about 1 μM, more specifically about 0.1 pM to about 100 nM. Methods for determining the affinity of interaction are known in the art. An antigen-specific extracellular domain suitable for use in a CAR may be any antigen-binding polypeptide, one or more scFv, or another antibody-based recognition domain (cAb VHH (camelid antibody variable domains) or humanized versions thereof, IgNAR VH (shark antibody variable domains) and humanized versions thereof, sdAb VH (single domain antibody variable domains) and “camelized” antibody variable domains are suitable for use. In some instances, T cell receptor (TCR) based recognition domains such as single chain TCR may be used as well as ligands for cytokine receptors.

The antigen recognition domain can bind a cell surface antigen found on a normal cell or a dysfunctional cell, such as a tumor-specific antigen. Exemplary cell surface antigens include B cell surface protein biomarkers such as CD19, CD20, CD34, CD38 and CD45R; senescent cell protein biomarkers such as uPAR, p16, p53 and p21; epithelial cell surface markers such as EpCAM; and additional normal cell surface markers such as CD29, CD9, CD166, CD44, Notch3 and CD123. CD19, for example, is a biomarker of both normal and neoplastic B cells.

The extracellular domain of the CAR can comprise an antigen recognition domain that binds a tumor-specific antigen.

The NK cells described herein express an antigen recognition domain that binds a tumor-specific antigen. The antigen can be expressed as a peptide or as an intact protein or portion thereof. The intact protein or a portion thereof can be native or mutagenized. Non-limiting examples of tumor-specific antigens that are CAR targets include wherein the tumor-specific antigen comprises carbonic anhydrase IX (CAIX), carcinoembryonic antigen (CEA), CD5, CD8, CD7, CD10, CD19, CD20, CD22, CD30, CD33, CLL1, CD34, CD38, CD41, CD44, CD49f, CD56, CD74, CD133, CD138, CD123, CD44V6, Claudin-18, B7 homolog 3 protein (B7-H3), fibroblast activation protein (FAP), cancer antigen 19 (CA19), an antigen of a cytomegalovirus (CMV) infected cell, epithelial glycoprotein-2 (EGP-2), epithelial glycoprotein-40 (EGP-40), epithelial cell adhesion molecule (EpCAM), receptor tyrosine-protein kinases erb-B2,3,4 (erb-B2,3,4), folate-binding protein (FBP), fetal acetylcholine receptor (AChR), adult AChR subunits, folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Ra2), κ-light chain, kinase insert domain receptor (KDR), Lewis Y (LeY), L1 cell adhesion molecule (LlCAM), melanoma antigen family A, 1 (MAGE-A1), Mucin 16 (MUC16), Mucin 1 (MUC1), Mesothelin (MSLN), PSMA, GPC3, ERBB2, MAGEA3, p53, MART1, GP100, Proteinase3 (PR1), Tyrosinase, Survivin, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGFR, EGFR-vIII, CD99, CD70, ADGRE2, CCR1, LILRB2, PRAME CCR4, CD5, CD3, TRBC1, TRBC2, TIM-3, Integrin B7, ICAM-1, CD70, Tim3, CLEC12A, ERBB, or a combination thereof.

Specific tumor-specific antigens include GD2, HER2, EGFR, mesothelin, Claudin-18.2, PSMA, B7-H3, IL-13Ra2, FAP, CA19, CD19, CD5, MUC1, or a combination thereof.

The use of CARs is not limited to cancer therapy. CAR NK cells can be used in treatments wherein a population of cells can be specifically targeted and eliminated to provide a therapeutic effect. In another aspect, the antigen can include an antigen such as autoimmune and inflammatory disease antigens such as antigens including B-cell and plasma lineage antigens such as BCMA, GPRC5D, CD20, CD22; cardiac disease antigens such as fibroblast-specific antigens and fibroblast activation protein (FAP), and LRRC15; senescence-associated disease ligands such as NKG2D ligands, MICA, MICB, ULBP1-5, glycoprotein non-metastatic melanoma protein B(GPNMB); other disease-associated antigens such as antigens associated with pulmonary aspergillosis; and the like.

The antigen-specific extracellular domain can also include a spacer linking the Vh and VL chains of the scFV, which can be the hinge region of IgG1 and is sufficient for most scFv-based constructs. Flexible linkers include glycine-serine linkers and Whitlow linkers.

The intracellular domain transmits the immune cell activation signal. The intracellular domain can increase immune cell cytokine production and facilitate immune cell replication. The intracellular domain reduces CAR NK cell exhaustion, increases NK cell antitumor activity, and enhances survival of CAR NK cells in patients. Exemplary intracellular domains comprise co-stimulatory domains, including those from CD27, CD28, CD137 or 4-1BB, CD154 or CD40L, CD244 or 2B4, CD278 or ICOS, CD134 or OX40, CD3-ζ, and combinations thereof, and signaling domains (also called cytotoxicity domains), including those from CD16, DAP10, DAP12, CD28, ICOS, CD27, OX40, CD40L, CD3-ζ, and combinations thereof. A costimulatory domain is derived from the intracellular signaling domains of costimulatory proteins that enhance cytokine production, proliferation, cytotoxicity, and/or persistence in vivo.

Typically, the antigen-specific extracellular domain is linked to the intracellular domain of the CAR by a transmembrane domain, e.g., derived from a CD4, CD8, CD-8 alpha, CD8-beta, CD3-epsilon, CD3-beta, CD28, 4-1BB, OC40, PD-1, LAG-3, CH2CH3 or NKG2D, IgG, CD3-ζ transmembrane domain, or combinations thereof. The transmembrane domain traverses the cell membrane, anchors the CAR to the NK cell surface, and connects the extracellular domain to the intracellular signaling domain, thus impacting expression of the CAR on the NK cell surface.

CARs may also further comprise one or more spacers. A spacer or hinge connects (i) the antigen-specific extracellular domain to the transmembrane domain, (ii) the transmembrane domain to a costimulatory domain, (iii) a costimulatory domain to the intracellular domain, and/or (iv) the transmembrane domain to the intracellular domain. For example, inclusion of a spacer domain (e.g., IgG1, IgG2, IgG4, CD28, CD8) between the antigen-specific extracellular domain and the transmembrane domain may affect flexibility of the antigen-binding domain and thereby CAR function. Transmembrane domains, costimulatory domains, and spacers are known in the art.

### Methods of Making Genetically Modified NK Cells

In an aspect, an ex vivo, non-viral method of site-specifically inserting a transgene containing a chimeric antigen receptor (CAR) gene into an endogenous NK cell gene, to provide genetically modified NK cells, wherein the endogenous NK cell gene encodes an NKG2A receptor, comprises
providing a non-viral homology-directed repair (HDR) template comprising the transgene flanked by homology arms that are complementary to sequences on both sides of a cleavage site in the endogenous NK cell gene, wherein the CAR comprises an extracellular domain linked to an intracellular domain through a first transmembrane domain, wherein the first extracellular domain comprises an antigen recognition domain that binds a tumor-specific antigen, and wherein cleavage at the cleavage site inactivates the NKG2A receptor; and
introducing into a population of unmodified NK cells a Cas9 ribonucleoprotein (RNP) and the double-stranded HDR template, to provide the genetically modified NK cells
   wherein the Cas9 RNP comprises a Cas9 protein and a guide RNA that directs double stranded DNA cleavage of the cleavage site in the endogenous NK gene, and
   wherein, after introducing the Cas9 RNP and the HDR template, the endogenous NK cell gene is knocked-out and the transgene is specifically knocked-in to the knock-out site, to provide the genetically modified NK cells.

In the methods described herein, a transgene containing a chimeric antigen receptor (CAR) gene is site-specifically inserted into the genome of an NK cell, specifically into the endogenous NK cell gene encoding an NKG2A receptor. CAR refers to a recombinant fusion protein that has an antigen-specific extracellular domain coupled to an intracellular domain that directs the cell to perform a specialized function upon binding of an antigen to the extracellular domain. In an aspect, a CAR comprises an antigen-specific extracellular domain (e.g., a single chain variable fragment [scFV] that can bind a surface-expressed antigen of a malignancy) coupled to an intracellular domain (e.g., CD28, ICOS, CD27, 4-1BB, OX40, CD40L, or CD3-ζ) by a transmembrane domain (e.g., derived from a CD4, CD8a, CD28, IgG or CD3-ζ transmembrane domain).

In order to insert the CAR into the genome of the unmodified NK cells, a Cas9 RNP and a non-viral HDR template including the transgene encoding the CAR are introduced into the unmodified NK cells to provide genome-edited NK cells.

As used herein, “introducing” means refers to the translocation of the Cas9 ribonucleoprotein and a non-viral HDR template from outside a cell to inside the cell, such as inside the nucleus of the cell. Introducing can include nucleofection, transfection, electroporation, contact with nanowires or nanotubes, receptor mediated internalization, translocation via cell penetrating peptides, liposome mediated translocation, and the like.

Genome editing of the NK cells as described herein uses a CRISPR system, or Cas9 ribonucleoprotein. CRISPR refers to the Clustered Regularly Interspaced Short Palindromic Repeats type II system used by bacteria and archaea for adaptive defense. This system enables bacteria and archaea to detect and silence foreign nucleic acids, e.g., from viruses or plasmids, in a sequence-specific manner. In type II systems, guide RNA interacts with Cas9 and directs the nuclease activity of Cas9 to target DNA sequences complementary to those present in the guide RNA. The Guide RNA binds with complementary sequences in target DNA. Cas9 nuclease activity then generates a double-stranded break in the target DNA.

CRISPR/Cas9 is a ribonucleoprotein (RNP) complex. CRISPR RNA (crRNA) includes a 20 base protospacer element that is complementary to a genomic DNA sequence as well as additional elements that are complementary to the transactivating RNA (tracrRNA). The tracrRNA hybridizes to the crRNA and binds to the Cas9 protein, to provide an active RNP complex. Thus, in nature, the CRISPR/Cas9 complex contains two RNA species.

Guide RNA, or gRNA, can be in the form of a crRNA/tracrRNA two guide system, or an single guide RNA (sgRNA). The guide RNA is capable of directing Cas9-mediated cleavage of target DNA. A guide RNA thus contains the sequences necessary for Cas9 binding and nuclease activity and a target sequence complementary to a target DNA of interest (protospacer sequence).

As used herein, a guide RNA protospacer sequence refers to the nucleotide sequence of a guide RNA that binds to a target genomic DNA sequence and directs Cas9 nuclease activity to a target DNA locus in the genome of the NK cell such the KLRC1 gene, AAVS1 (i.e., PPP1R12C), TIGIT, CISH, and PDCD1. In some embodiments, the guide RNA protospacer sequence is complementary to the target DNA sequence. “Complementary” or “complementarity” refers to specific base pairing between nucleotides or nucleic acids. Base pairing between a guide RNA and a target region in exon 3 of the KLRC1 gene can be via a DNA targeting sequence that is perfectly complementary or substantially complementary to the guide RNA. As described herein, the protospacer sequence of a single guide RNA may be customized, allowing the targeting of Cas9 activity to a target DNA of interest.

Any desired target DNA sequence of interest may be targeted by a guide RNA target sequence. Any length of target sequence that permits CRISPR-Cas9 specific nuclease activity may be used in a guide RNA. In some embodiments, a guide RNA contains a 20 nucleotide protospacer sequence.

In addition to the protospacer sequence, the targeted sequence includes a protospacer adjacent motif (PAM) adjacent to the protospacer region which is a sequence recognized by the CRISPR RNP as a cutting site. Without wishing to be bound to theory, it is thought that the only requirement for a target DNA sequence is the presence of a protospacer-adjacent motif (PAM) adjacent to the sequence complementary to the guide RNA target sequence. Different Cas9 complexes are known to have different PAM motifs. For example, Cas9 from Streptococcus pyogenes has a NGG trinucleotide PAM motif; the PAM motif of N. meningitidis Cas9 is NNNNGATT; the PAM motif of S. thermophilus Cas9 is NNAGAAW; and the PAM motif of T. denticola Cas9 is NAAAAC.

In an aspect, to target the endogenous NK cell gene encoding an NKG2A receptor, the guide RNA can comprise GGTCTGAGTAGATTACTCCT (SEQ ID NO: 1) and the PAM is TGG.

A “Cas9” polypeptide is a polypeptide that functions as a nuclease when complexed to a guide RNA, e.g., an sgRNA or modified sgRNA. That is, Cas9 is an RNA-mediated nuclease. The Cas9 (CRISPR-associated 9, also known as Csn1) family of polypeptides, for example, when bound to a crRNA:tracrRNA guide or single guide RNA, are able to cleave target DNA at a sequence complementary to the sgRNA target sequence and adjacent to a PAM motif as described above. Cas9 polypeptides are characteristic of type II CRISPR-Cas systems. The broad term “Cas9” Cas9 polypeptides include natural sequences as well as engineered Cas9 functioning polypeptides. The term “Cas9 polypeptide” also includes the analogous Clustered Regularly Interspaced Short Palindromic Repeats from Prevotella and Francisella 1 or CRISPR/Cpf1 which is a DNA-editing technology analogous to the CRISPR/Cas9 system. Cpf1 is an RNA-guided endonuclease of a class II CRISPR/Cas system. This acquired immune mechanism is found in Prevotella and Francisella bacteria. Additional Class I Cas proteins include Cas3, Cas8a, Cas5, Cas8b, Cas8c, Cas 10d, Case1, Cse 2, Csy 1, Csy 2, Csy 3, GSU0054, Cas 10, Csm 2, Cmr 5, Cas10, Csx11, Csx10, and Csf 1. Additional Class 2 Cas9 polypeptides include Csn 2, Cas4, C2c1, C2c3 and Cas13a.

Exemplary Cas9 polypeptides include Cas9 polypeptide derived from Streptococcus pyogenes, e.g., a polypeptide having the sequence of the Swiss-Prot accession Q99ZW2 (SEQ ID NO: 65); Cas9 polypeptide derived from Streptococcus thermophilus, e.g., a polypeptide having the sequence of the Swiss-Prot accession G3ECR1 (SEQ ID NO: 66); a Cas9 polypeptide derived from a bacterial species within the genus Streptococcus; a Cas9 polypeptide derived from a bacterial species in the genus Neisseria (e.g., GenBank accession number YP_003082577; WP_015815286.1 (SEQ ID NO: 67)); a Cas9 polypeptide derived from a bacterial species within the genus Treponema (e.g., GenBank accession number EMB41078 (SEQ ID NO: 68)); and a polypeptide with Cas9 activity derived from a bacterial or archaeal species. Methods of identifying a Cas9 protein are known in the art. For example, a putative Cas9 protein may be complexed with crRNA and tracrRNA or sgRNA and incubated with DNA bearing a target DNA sequence and a PAM motif.

The term “Cas9” or “Cas9 nuclease” refers to an RNA-guided nuclease comprising a Cas9 protein, or a fragment thereof (e.g., a protein comprising an active, inactive, or partially active DNA cleavage domain of Cas9, and/or the gRNA binding domain of Cas9). In some embodiments, a Cas9 nuclease has an inactive (e.g., an inactivated) DNA cleavage domain, that is, the Cas9 is a nickase. Other embodiments of Cas9, both DNA cleavage domains are inactivated. This is referred to as catalytically-inactive Cas9, dead Cas9, or dCas9.

Functional Cas9 mutants are described, for example, in US20170081650 and US20170152508, incorporated herein by reference for its disclosure of Cas9 mutants.

As used herein, the term editing refers to a change in the sequence of the genome at a targeted genomic location. Editing can include inducing either a double stranded break or a pair of single stranded breaks in the genome, such as in a NK cell expressed gene. Editing can also include inserting a synthetic DNA sequence into the genome of the NK cell at the site of the break(s).

As used herein, a Cas9 RNP that targets the endogenous NK cell gene encoding an NKG2A receptor comprises a Cas9 protein and a guide RNA that directs double stranded cleavage of the gene encoding an NKG2A receptor. The guide RNA thus includes a crRNA comprising a single-stranded protospacer sequence and a first complementary strand of a binding region for the Cas9 polypeptide, and a tracrRNA comprising a second complementary strand of the binding region for the Cas9 polypeptide, wherein the crRNA and the tracrRNA hybridize through the first and second complementary strands of the binding region for the Cas9 polypeptide. The single-stranded protospacer region of the guide RNA hybridizes to a sequence in the gene encoding the NKG2A receptor, directing cleavage of the gene encoding the NKG2A receptor to a specific locus of the gene encoding the NKG2A receptor.

In addition to the Cas9 RNP, a non-viral HDR template, e.g. a double-stranded or single-stranded HDR template, comprising the CAR transgene is introduced into the NK cells. In prior art methods, viral vectors such as adeno-associated virus vectors have been used to provide the CAR transgene. Even when combined with Cas9 RNP gene editing, the use of AAV vectors (a) are expensive; (b) could integrate viral genomes into the human genome; (c) trigger an immune response within the patient to viral components; (d) may result in highly variable transgene expression; and (d) take extended periods of time (e.g., months to years) to manufacture.

In an aspect, the non-viral HDR template comprises the transgene flanked by homology arms that are complementary to sequences on both sides of a cleavage site in the endogenous NK cell gene. The homology arms have 50 to 3000 nucleotides in length and are complementary to sequences on either side of the cut site in the NK cell expressed gene to facilitate incorporation of the synthetic DNA sequence into the genome of the NK cell. Small sequence variations (<100 bases) from complementary sequences could be included to enable barcoding or tracking of various cell types.

In an aspect, the length of the homology arms can improve the efficiency of synthetic DNA sequence integration. In an aspect, the homology arms are 400 to 1000 base pairs, specifically 450 to 750 base pairs long.

In an aspect, the non-viral HDR template sequentially comprises a left homology arm-a self-cleaving peptide sequence (e.g., a T2A coding sequence)-a CAR gene-a polyA terminator-a right homology arm. In another aspect, the non-viral HDR template sequentially comprises a left homology arm-a splice acceptor site-a self-cleaving peptide sequence (e.g., a T2A coding sequence)-a CAR gene-a polyA terminator-a right homology arm.

The optional splice acceptor site assists in the splicing of the synthetic DNA sequence into the transcript generated from the endogenous NK cell gene.

The self-cleaving peptide sequence, e.g., T2A, assists in the separation or cleavage of the translated peptide of the protein product encoded by the synthetic DNA sequence from the protein product of the native NK cell expressed gene. Exemplary self-cleaving peptides sequences include viral 2A peptides such as the porcine teschovirus-1 (P2A) peptide, a Thoseaasigna virus (T2A) peptide, an equine rhinitis A virus (E2A) peptide, or a foot-and-mouth disease virus (F2A) peptide.

The polyA terminator, e.g., a bovine growth hormone polyA. The polyA terminator is a sequence-based element that defines the end of a transcriptional unit within the synthetic DNA sequence and initiate the process of releasing the newly synthesized RNA from the transcription machinery.

In an aspect, the population of unmodified NK cells is expanded for 4-7 days prior to introducing the Cas9 ribonucleoprotein (RNP) and the HDR template. In an aspect, expansion is done in the presence of feeder cells.

In another aspect, introducing the Cas9 ribonucleoprotein (RNP) and the HDR template into the population of unmodified NK cells is done in the presence of a DNA-dependent protein kinase (DNA-PK) inhibitor, e.g., M3814.

In yet another aspect, introducing the Cas9 ribonucleoprotein (RNP) and the HDR template into the unmodified NK cells is done using an EH-100 pulse sequence (Lonza Amaxa™ 4D Nucleofector™ system).

After the genetically modified NK calls are produced, they can be further expanded to provide an expanded population of genetically modified NK cells.

### Methods of Treating and Pharmaceutical Compositions

In an aspect, the compositions and methods described herein are particularly useful to treat solid tumors such as a sarcoma, adrenocortical carcinoma, retinoblastoma, kidney cancer, bladder cancer, breast cancer, neuroblastoma, melanoma, sarcoma, neuroendocrine cancer, colorectal cancer, lung cancer, head and neck cancer, prostate cancer, pancreatic cancer, ovarian cancer, uterine cancer, oral cavity cancer, glioblastoma, lymphoma, diffuse midline glioma, carcinoid tumors, neuroendocrine tumors, thyroid cancer, liver cancer, or a combination thereof. Solid tumors can be primary or metastatic solid tumors. In an aspect, the solid tumor is a poorly immunogenic solid tumor.

“Pharmaceutically acceptable” as used herein means that the compound or composition or carrier is suitable for administration to a subject to achieve the treatments described herein, without unduly deleterious side effects in light of the necessity of the treatment.

The term “effective amount,” as used herein, refers to the amount of the compounds or dosages that will elicit the biological or medical response of a subject, tissue or cell that is being sought by the researcher, veterinarian, medical doctor or other clinician.

The term, “pharmaceutically-acceptable carrier” includes any and all dry powder, solvents, dispersion media, coatings, antibacterial and antifungal agents, isotonic agents, absorption delaying agents, and the like. Pharmaceutically-acceptable carriers are materials, useful for the purpose of administering the compounds in the method of the present invention, which are preferably non-toxic, and may be solid, liquid, or gaseous materials, which are otherwise inert and pharmaceutically acceptable, and are compatible with the compounds described herein. Examples of such carriers include, without limitation, various lactose, mannitol, oils such as corn oil, buffers such as PBS, saline, polyethylene glycol, glycerin, polypropylene glycol, dimethylsulfoxide, an amide such as dimethylacetamide, a protein such as albumin, and a detergent such as Tween 80, mono- and oligopolysaccharides such as glucose, lactose, cyclodextrins and starch.

The term “administering” or “administration,” as used herein, refers to providing the compound or pharmaceutical composition of the invention to a subject suffering from or at risk of the diseases or conditions to be treated or prevented.

A route of administration in pharmacology is the path by which a drug is taken into the body. Routes of administration may be generally classified by the location at which the substance is applied. Common examples may include oral and intravenous administration. Routes can also be classified based on where the target of action is. Action may be topical (local), enteral (system-wide effect, but delivered through the gastrointestinal tract), or parenteral (systemic action, but delivered by routes other than the GI tract), via lung by inhalation. One form of local administration is intratumoral (IT), whereby an agent is injected directly into, or adjacent to, a known tumor site.

A topical administration emphasizes local effect, and substance is applied directly where its action is desired. Sometimes, however, the term topical may be defined as applied to a localized area of the body or to the surface of a body part, without necessarily involving target effect of the substance, making the classification rather a variant of the classification based on application location. In an enteral administration, the desired effect is systemic (non-local), substance is given via the digestive tract. In a parenteral administration, the desired effect is systemic, and substance is given by routes other than the digestive tract.

Examples of parenteral administrations may include intravenous (into a vein), e.g. many drugs, total parenteral nutrition intra-arterial (into an artery), e.g., vasodilator drugs in the treatment of vasospasm and thrombolytic drugs for treatment of embolism, intraosseous infusion (into the bone marrow), intra-muscular, intracerebral (into the brain parenchyma), intracerebroventricular (into cerebral ventricular system), intrathecal (an injection into the spinal canal), and subcutaneous (under the skin). Among them, intraosseous infusion is, in effect, an indirect intravenous access because the bone marrow drains directly into the venous system. Intraosseous infusion may be occasionally used for drugs and fluids in emergency medicine and pediatrics when intravenous access is difficult.

The methods can further comprise administering a therapeutic antibody such as hu14.18K322A (a humanized antidisialoganglioside monoclonal antibody), trastuzumab, cetuximab, naxitamab, amivantarnab, panitumumab, necitununab, bevacizumab, ramucirumab, pertuzumab, ado-trastuzumab emtansine, dinutuximab, relatimab, margetuximab, or a combination thereof.

The invention is further illustrated by the following non-limiting examples.

### EXAMPLES

### Methods

Cell Lines: M21 human melanoma cells were a gift from Dr. Paul Sondel (University of Wisconsin-Madison), and K562-mb15-41BBL cells were a gift from Dr. Christian Capitini (University of Wisconsin-Madison). Both cell lines were maintained in RPMI 1640 (Gibco) supplemented with 10% fetal bovine serum (FBS) (Cytiva), 1% penicillin-streptomycin (Gibco), and 1% L-glutamine (Gibco). NK-92 cells (ATCC, Manassas, VA) were cultured in X-VIVO 10 (Lonza, Basel, Switzerland) supplemented with 5% FBS (WiCell, Madison, WI), 5% horse serum (ThermoFisher, Waltham, MA), 199.82 —M myo-inositol (Sigma Aldrich, St. Louis, MO), and 19.94 μM folic acid (Sigma Aldrich, St. Louis, MO). The M21-HLA-E transduced cell line was maintained in the same medium with the addition of 600 μg/mL of G418 Geneticin. Cell authentication was performed using short tandem repeat analysis (Idexx BioAnalytics, Westbrook, Maine) and per ATCC guidelines using cell morphology, growth curves, and Mycoplasma testing within 6 months with the MycoStrip Mycoplasma Detection Kit (Invitrogen, Waltham, MA). Cell lines were maintained in culture at 37° C. in 5% CO₂.

The human HLA-E gene insert (Table 1), incorporating the HLA-G leader peptide, and HindIII and EcoRI restriction sites, was generated synthetically by IDT (IDT, Newark, NJ). The pcDNA™3.1(+) plasmid (ThermoFisher, Waltham, MA) and the synthesized HLA-E gene were digested separately using HindIII and EcoRI enzymes (ThermoFisher, Waltham, MA) following the manufacturer's instructions. The digested products (backbone of pcDNA™3.1 and HLA-E gene product) were ligated using the T4 DNA ligase enzyme (ThermoFisher, Waltham, MA) according to the manufacturer's instructions. The ligated product was transformed into JM109 competent E. coli. (Promega, Madison, WI) using a heat shock protocol. To confirm successful transformation of clones, DNA sequencing was performed using primers specific to the pcDNA3.1 CMV (forward) and BGH (reverse) sequences.

24 hours before transfection, M21 cells (1×10⁶ cells per well) were plated in a 6 well plate, with cells reaching 80-90% confluency at the time of transfection. Cell transfection was performed using the TransIT-Lenti transfection reagent (Mirus bio, Madison, WI) per the manufacturer's instructions. After 24 hours of transfection, the cells were treated with 600 μg/mL of G418 Geneticin (ThermoFisher, Waltham, MA) to select for transfected cells. Monoclonal cell populations were generated through a serial dilution technique, and flow cytometry was used to select the clone with the highest HLA-E expression.

Isolation and expansion of NK cells: Whole blood was collected from the median cubital vein of healthy donors using 10 mL lithium heparin-coated vacutainers (158 USP units, BD Biosciences). Peripheral blood NK cells were isolated from whole blood using a density based negative selection kit according to the manufacturer's instructions (RosetteSep™ Human NK Enrichment Cocktail, STEMCELL Technologies). Isolated NK cells were cultured with irradiated K562-mb15-41BBL (1-3 days before NK isolation, 100 Gy) along with 50 IU/mL IL-2 (Peprotech) and 2 ng/mL IL-15 (Peprotech) at a ratio of 1:2 effort:target (E:T) in NK MACS medium (Miltenyi) supplemented with 5% human AB serum (Millipore Sigma) and 1% MACS® supplement (Miltenyi). Total density was maintained at 1e6 cells/mL. Cells were cultured until day 4, after which they were nucleofected for experiments. After day 5, unedited and nucleofected NK cells were cultured in complete NK MACS medium (Miltenyi Biotec, Gaithersburg, MD) with 100 IU/mL IL-2 and 2 ng/mL IL-15. Cells were maintained at a density of 1e6 cells/mL and were passaged every two days. Feeder cells were added to support NK expansion on days 0 and 5.

spCas9 RNP complexing: Purified sNLS-SpCas9-sNLS Cas9 protein (10 mg/mL) was purchased from Aldevron. The sgRNA was ordered from IDT with the crRNA and tracrRNA pre-complexed. 60 pmoles of sgRNA (IDT) was aliquoted into individual PCR tubes for each nucleofection replicate. Sterile-filtered 15-50 kDa poly(L-glutamic acid) (PGA) (Sigma Aldrich) was added to the sgRNA at 0.8:1 volume ratio as previously described in the art. 50 pmoles of Cas9 was added to the sgRNA+PGA mixture for a final molar ratio of 1:1.2 sgRNA:Cas9. The complexes were incubated at 37° C. for 15 minutes prior to use.

Preparation of dsDNA templates: PCR amplicons of the CAR and mCherry transgenes were generated as described previously in the art. PCR products were pooled into 600 μL and were purified using a singular round of Solid Phase Reversible Immobilization (SPRI) cleanup using AMPure XP beads (Beckman Coulter) per the manufacturer's instructions, at a 1:1 ratio. Bead incubation and elution times were increased to 5 minutes and 15 minutes, respectively. Each pooled sample was eluted in 30 μL of nuclease-free water (IDT), which were then combined to form one sample. 3M pH5.5 sodium acetate (NaOAc) (Sigma Aldrich) was added to the pooled sample at 0.1× of the total volume. The sample was ethanol precipitated by adding 2.5× of 100% ethanol, followed by a 30-minute incubation at −80° C. The precipitate was purified using an ethanol wash (70-80%) and eluted in 20-30 μL of nuclease free water. DNA template concentration was measured and adjusted as previously described in the art. DNA sequences are listed in Table 1 and primer sequences are listed in Table 2.

TABLE 1
DNA sequences

| | SEQ | |
|---|---|---|
| | ID | |
| Identity | NO: | Sequence |
| gRNA1 (PAM) | 1 | GGTCTGAGTAGATTACTCCT(TGG) |
| gRNA2 (PAM) | 2 | TGAACAGGAAATAACCTATG(CGG) |
| gRNA3 (PAM) | 3 | TCTTATCTTAATGGCCTCTG(TGG) |
| Left | 4 | AACTTCTTTATTCCTTAGTAATTTATTGCTTCTCATTGCCCCAGC |
| Homology | | AATAATATTTTGTCAAATGCAGAAAAtttatcttttttttttgagtcggag |
| Arm | | tctcactctgtcgcccaggctggagtgcagtggcacaatctccgctcactgcaacct |
| | | ctgccgcccatgttcaagagattctcctgcctcagcctcccgagtagctaggactac |
| | | aggcgcctgacatcatgcccgactactttttgtatttttagtagagacggagtttcac |
| | | cgtgttagccaggatggtctcgatctcctgacctcgtgatcggcatgcctcggcctc |
| | | ccaaagtgctgggattacaggcgtgagccaccgcgcccggccTAAAAATCTT |
| | | TTTTTAAAACAAATATTCATAAGAAACGTGTTTAGGCTTGAAG |
| | | AAAATCAGAGAAAGAACTTTAGATTATTTAATGCAAAATGAGC |
| | | TCCAATACTCGTTCTCCACCTCACCCTTTTAATTGCACTAGGGA |
| | | ATCCTGTATATAAACCATTTATTAACTTCTTAACTACTGTTATTA |
| | | TAGAGTACAGTCCCTGACATCACACACTGCAGAGATGGATAAC |
| | | CAAGG |
| Right | 5 | AGTAATCTACTCAGACCTGAATCTGCCCCCAAACCCAAAGAGG |
| homology | | CAGCAACGAAAACCTAAAGGCAATAAAAACTCCATTTTAGCAA |
| Arm | | CTGAACAGGAAATAACCTATGCGGAATTAAACCTTCAAAAAGC |
| | | TTCTCAGGATTTTCAAGGGAATGACAAAACCTATCACTGCAAA |
| | | GGTAAAGCATTTAAAAGATCCTCAATATAACAGTCTAGGATGT |
| | | GCAGCTTGGGGTACAGGAATGTGGGGAAAGAGAAGGGAGTG |
| | | CTCATATATCTTCTATTTGCAAAGATCAGAATTCCAAGTTGAGA |
| | | TATGCTATTTCAATGTAAAGTATGAAGACTGATTGAACTCATTG |
| | | TTGAAGTTTGTAGTCTTTGTCAAATAATTCATGGAGCATTATTT |
| | | TTCCTGAAAATTCAATGGTATATTATTCTGAGAAAAAGATTACA |
| | | ATGGGAGATGAGGGTTTGGGGTCCAAGTTTCTCTGTATGATTC |
| | | CTGTGCATTCAGGTTCTCTTGTCTGTGAATCTTCTAAACGACTG |
| | | TATCCACCTCTCCTTTCGCACTGTTCCCATTTCTCTCCCTGCAGA |
| | | TTTACCATCAGCTCCAGAGAAGCTCATTGTTGGGAT |
| CAR | 6 | Aggcagcggagagggcagaggaagtcttctaacatgcggtgacgtggaggaga |
| | | atcccggccctATGGAGTTTGGGCTGAGCTGGCTTTTTCTTGTGGC |
| | | TATTTTAAAAGGTGTCCAGTGCTCTAGAGATATTTTGCTGACCC |
| | | AAACTCCACTCTCCCTGCCTGTCAGTCTTGGAGATCAAGCCTCC |
| | | ATCTCTTGCAGATCTAGTCAGAGTCTTGTACACCGTAATGGAA |
| | | ACACCTATTTACATTGGTACCTGCAGAAGCCAGGCCAGTCTCC |
| | | AAAGCTCCTGATTCACAAAGTTTCCAACCGATTTTCTGGGGTCC |
| | | CAGACAGGTTCAGTGGCAGTGGATCAGGGACAGATTTCACAC |
| | | TCAAGATCAGCAGAGTGGAGGCTGAGGATCTGGGAGTTTATT |
| | | TCTGTTCTCAAAGTACACATGTTCCTCCGCTCACGTTCGGTGCT |
| | | GGGACCAAGCTGGAGCTGAAACGGGCTGATGCTGCACCAACT |
| | | GTATCCATCTTCCCAGGCTCGGGCGGTGGTGGGTCGGGTGGC |
| | | GAGGTGAAGCTTCAGCAGTCTGGACCTAGCCTGGTGGAGCCT |
| | | GGCGCTTCAGTGATGATATCCTGCAAGGCTTCTGGTTCCTCATT |
| | | CACTGGCTACAACATGAACTGGGTGAGGCAGAACATTGGAAA |
| | | GAGCCTTGAATGGATTGGAGCTATTGATCCTTACTATGGTGGA |
| | | ACTAGCTACAACCAGAAGTTCAAGGGCAGGGCCACATTGACT |
| | | GTAGACAAATCGTCCAGCACAGCCTACATGCACCTCAAGAGCC |
| | | TGACATCTGAGGACTCTGCAGTCTATTACTGTGTAAGCGGAAT |
| | | GGAGTACTGGGGTCAAGGAACCTCAGTCACCGTCTCCTCAGCC |
| | | AAAACGACACCCCCATCAGTCTATGGAAGGGTCACCGTCTCTT |
| | | CAGCGGAGCCCAAATCTTGTGACAAAACTCACACATGCCCACC |
| | | GTGCCCGGATCCCAAATTTTGGGTGCTGGTGGTGGTTGGTGG |
| | | AGTCCTGGCTTGCTATAGCTTGCTAGTAACAGTGGCCTTTATTA |
| | | TTTTCTGGGTGAGGAGTAAGAGGAGCAGGCTCCTGCACAGTG |
| | | ACTACATGAACATGACTCCCCGCCGCCCCGGGCCCACCCGCAA |
| | | GCATTACCAGCCCTATGCCCCACCACGCGACTTCGCAGCCTATC |
| | | GCTCCAAACGGGGCAGAAAGAAACTCCTGTATATATTCAAACA |
| | | ACCATTTATGAGACCAGTACAAACTACTCAAGAGGAAGATGGC |
| | | TGTAGCTGCCGATTTCCAGAAGAAGAAGAAGGAGGATGTGAA |
| | | CTGAGAGTGAAGTTCAGCAGGAGCGCAGACGCCCCCGCGTAC |
| | | CAGCAGGGCCAGAACCAGCTCTATAACGAGCTCAATCTAGGA |
| | | CGAAGAGAGGAGTACGATGTTTTGGACAAGAGACGTGGCCG |
| | | GGACCCTGAGATGGGGGGAAAGCCGAGAAGGAAGAACCCTC |
| | | AGGAAGGCCTGTACAATGAACTGCAGAAAGATAAGATGGCGG |
| | | AGGCCTACAGTGAGATTGGGATGAAAGGCGAGCGCCGGAGG |
| | | GGCAAGGGGCACGATGGCCTTTACCAGGGTCTCAGTACAGCC |
| | | ACCAAGGACACCTACGACGCCCTTCACATGCAGGCCCTGCCCC |
| | | CTcgcTAATTCACTCCTCAGGTGCAGGCTGCCTATCAGAAGGTG |
| | | GTGGCTGGTGTGGCCAATGCCCTGGCTCACAAATACCACTGAG |
| | | ATCTTTTTCCCTCTGCCAAAAATTATGGGGACATCATGAAGCCC |
| | | CTTGAGCATCTGACTTCTGGCTAATAAAGGAAATTTATTTTCAT |
| | | TGCAATAGTGTGTTGGAATTTTTTGTGTCTCTCACTCGGAAGG |
| | | ACATATGGGAGGGCAAATCATTTAAAACATCAGAATGAGTATT |
| | | TGGTTTAGAGTTTGGCAACATATGCCCATATGCTGGCTGCCAT |
| | | GAACAAAGGTTGGCTATAAAGAGGTCATCAGTATATGAAACA |
| | | GCCCCCTGCTGTCCATTCCTTATTCCATAGAAAAGCCTTGACTT |
| | | GAGGTTAGATTTTTTTTATATTTTGTTTTGTGTTATTTTTTTCTTT |
| | | AACATCCCTAAAATTTTCCTTACATGTTTTACTAGCCAGATTTTT |
| | | CCTCCTCTCCTGACTACTCCCAGTCATAGCTGTCCCTCTTCTCTT |
| | | ATGGAGATCCCTCGACCTGCAGCCCAAGCTTGGCGTAATCATG |
| | | GTCATAGCTGT |
| mCherry | 7 | Aggcagcggagagggcagaggaagtcttctaacatgcggtgacgtggaggaga |
| | | atcccggccctATGCCTGAACCCTCTAAGTCTGCTCCAGCCCCTAA |
| | | AAAGGGTTCTAAGAAGGCTATCACTAAGGCGCAGAAGAAGGA |
| | | TGGTAAGAAGCGTAAGCGCAGCCGCAAGGAGAGCTATTCTAT |
| | | CTATGTGTACAAGGTTCTGAAGCAGGTCCACCCCGACACCGGC |
| | | ATCTCATCCAAGGCCATGGGGATCATGAACTCCTTCGTCAACG |
| | | ACATCTTCGAGCGCATCGCGGGCGAGGCTTCTCGCCTGGCTCA |
| | | CTACAATAAGCGCTCGACCATCACCTCCAGGGAGATTCAGACG |
| | | GCTGTGCGCCTGCTGCTGCCTGGGGAGCTGGCTAAGCATGCT |
| | | GTGTCCGAGGGCACTAAGGCAGTTACCAAGTACACTAGCTCTA |
| | | AGGATCCACCGGTCGCCACCATGGTGAGCAAGGGCGAGGAG |
| | | GATAACATGGCCATCATCAAGGAGTTCATGCGCTTCAAGGTGC |
| | | ACATGGAGGGCTCCGTGAACGGCCACGAGTTCGAGATCGAGG |
| | | GCGAGGGCGAGGGCCGCCCCTACGAGGGCACCCAGACCGCC |
| | | AAGCTGAAGGTGACCAAGGGTGGCCCCCTGCCCTTCGCCTGG |
| | | GACATCCTGTCCCCTCAGTTCATGTACGGCTCCAAGGCCTACGT |
| | | GAAGCACCCCGCCGACATCCCCGACTACTTGAAGCTGTCCTTC |
| | | CCCGAGGGCTTCAAGTGGGAGCGCGTGATGAACTTCGAGGAC |
| | | GGCGGCGTGGTGACCGTGACCCAGGACTCCTCCCTGCAGGAC |
| | | GGCGAGTTCATCTACAAGGTGAAGCTGCGCGGCACCAACTTCC |
| | | CCTCCGACGGCCCCGTAATGCAGAAGAAGACCATGGGCTGGG |
| | | AGGCCTCCTCCGAGCGGATGTACCCCGAGGACGGCGCCCTGA |
| | | AGGGCGAGATCAAGCAGAGGCTGAAGCTGAAGGACGGCGGC |
| | | CACTACGACGCTGAGGTCAAGACCACCTACAAGGCCAAGAAG |
| | | CCCGTGCAGCTGCCCGGCGCCTACAACGTCAACATCAAGTTGG |
| | | ACATCACCTCCCACAACGAGGACTACACCATCGTGGAACAGTA |
| | | CGAACGCGCCGAGGGCCGCCACTCCACCGGCGGCATGGACGA |
| | | GCTGTACAAGTAAttcactcctcaggtgcaggctgcctatcagaaggtggtg |
| | | gctggtgtggccaatgccctggctcacaaataccactgagatctttttccctctgcc |
| | | aaaaattatggggacatcatgaagccccttgagcatctgacttctggctaataaag |
| | | gaaatttattttcattgcaatagtgtgttggaattttttgtgtctctcactcggaagga |
| | | catatgggagggcaaatcatttaaaacatcagaatgagtatttggtttagagtttg |
| | | gcaacatatgcccatatgctggctgccatgaacaaaggttggctataaagaggtc |
| | | atcagtatatgaaacagccccctgctgtccattccttattccatagaaaagccttga |
| | | cttgaggttagattttttttatattttgttttgtgttatttttttctttaacatccctaaaa |
| | | ttttccttacatgttttactagccagatttttcctcctctcctgactactcccagtcata |
| | | gctgtccctcttctcttatggagatccctcgacctgcagcccaagcttggcgtaatc |
| | | atggtcatagctgt |
| HLA-E | 8 | AAGCTTATGGTGGTCATGGCGCCCCGAACCCTCTT |
| | | CCTGCTGCTCTCGGGGGCCCTGACCCTGACCGAGA |
| | | CCTGGGCGGGCTCCCACTCCTTGAAGTATTTCCAC |
| | | ACTTCCGTGTCCCGGCCCGGCCGCGGGGAGCCCCG |
| | | CTTCATCTCTGTGGGCTACGTGGACGACACCCAGT |
| | | TCGTGCGCTTCGACAACGACGCCGCGAGTCCGAGG |
| | | ATGGTGCCGCGGGCGCCGTGGATGGAGCAGGAGG |
| | | GGTCAGAGTATTGGGACCGGGAGACACGGAGCGC |
| | | CAGGGACACCGCACAGATTTTCCGAGTGAACCTGC |
| | | GGACGCTGCGCGGCTACTACAATCAGAGCGAGGCC |
| | | GGGTCTCACACCCTGCAGTGGATGCATGGCTGCGA |
| | | GCTGGGGCCCGACAGGCGCTTCCTCCGCGGGTATG |
| | | AACAGTTCGCCTACGACGGCAAGGATTATCTCACC |
| | | CTGAATGAGGACCTGCGCTCCTGGACCGCGGTGGA |
| | | CACGGCGGCTCAGATCTCCGAGCAAAAGTCAAATG |
| | | ATGCCTCTGAGGCGGAGCACCAGAGAGCCTACCTG |
| | | GAAGACACATGCGTGGAGTGGCTCCACAAATACCT |
| | | GGAGAAGGGGAAGGAGACGCTGCTTCACCTGGAG |
| | | CCCCCAAAGACACACGTGACTCACCACCCCATCTC |
| | | TGACCATGAGGCCACCCTGAGGTGCTGGGCCCTGG |
| | | GCTTCTACCCTGCGGAGATCACACTGACCTGGCAG |
| | | CAGGATGGGGAGGGCCATACCCAGGACACGGAGC |
| | | TCGTGGAGACCAGGCCTGCAGGGGATGGAACCTTC |
| | | CAGAAGTGGGCAGCTGTGGTGGTGCCTTCTGGAGA |
| | | GGAGCAGAGATACACGTGCCATGTGCAGCATGAG |
| | | GGGCTACCCGAGCCCGTCACCCTGAGATGGAAGCC |
| | | GGCTTCCCAGCCCACCATCCCCATCGTGGGCATCA |
| | | TTGCTGGCCTGGTTCTCCTTGGATCTGTGGTCTCTG |
| | | GAGCTGTGGTTGCTGCTGTGATATGGAGGAAGAAG |
| | | AGCTCAGGTGGAAAAGGAGGGAGCTACTCTAAGG |
| | | CTGAGTGGAGCGACAGTGCCCAGGGGTCTGAGTCT |
| | | CACAGCTTGTAAGAATTC |

TABLE 2
Primer sequences for dsDNA template
preparation (5′ to 3′)

| Identity | SEQ ID NO: | Sequence |
|---|---|---|
| Forward | 9 | cggagtctcactctgtcgccca |
| Reverse | 10 | TGGACCCCAAACCCTCATCTCCC |

Nucleofection: NK-cells were nucleofected on day 4, unless otherwise described for optimization experiments. Post-nucleofection recovery medium (Complete NK MACS® medium (Miltenyi) with 100 IU/mL IL-2 (Peprotech) and 2 ng/mL IL-15 (Peprotech)+/−0.6 uM M3814 (Selleck Chem)) was prepared, with M3814 added at 1.2× the working concentration to account for future dilution. For M3814 related optimization experiments, multiple aliquots of post-recovery medium were prepared with a corresponding concentration of M3814. 150 μL of M3814 recovery medium was plated into individual wells of a 96-well round bottom plate for each replicate and set aside in the incubator. PB-NK cells were harvested and counted using the Countess® II FL Automated Cell Counter (Thermo Fisher Scientific) with 0.4% Trypan Blue viability stain (Thermo Fisher Scientific). 5e5 cells were aliquoted into individual 1.5 mL Eppendorf Tubes® for each nucleofection replicate. While the RNP complexes incubated, the cells were pelleted at 100 g for 10 minutes, and 3 μg (1.5 μL) of dsDNA templates were aliquoted into PCR tubes for each replicate. Following incubation, the RNP complexes were added to the dsDNA templates and were allowed to incubate for 3-5 minutes at room temperature. Cell pellets were resuspended in P3 buffer with 22% supplement (Lonza), and combined with the RNP+dsDNA mixture, for a total volume of 24 L. Samples were added to the 24 μL reaction cuvettes (Lonza) and were nucleofected using the EH-100 program (Lonza Amaxa™ 4D-Nucleofector®), unless specified otherwise for optimization experiments. Immediately after nucleofection, 100 μL of pre-warmed recovery medium (no M3814) was added to each of the cuvette wells, and samples were rested at 37° C. for 15 minutes. Finally, samples were transferred from the cuvette to the 96-well plate containing pre-warmed recovery medium with M3814. After 24 hours, nucleofected NK cells were combined with irradiated K562-mb15-41BBL (100 Gy) at a 1:2 ratio in complete NK MACS medium with 100 IU/mL IL-2 and 2 ng/mL IL-15.

NK cell culture: After day 5, unedited and nucleofected NK cells were cultured in complete NK MACS® medium (Miltenyi) with 100 IU/mL IL-2 (Peprotech) and 2 ng/mL IL-15 (Peprotech). Cells were maintained at a density of 1e6 cells/mL and were passaged every two days. Irradiated K562-mb15-41BBL (100 Gy) cells were only used to supplement NK expansion on days 0 and 5.

Flow cytometry and cell sorting: 1e5 cells were plated per condition and stained in 96-well round bottom plates. Cells were washed with 1×PBS, centrifuged at 1200 g for 1 minute, and stained with 0.1 μL/mL Zombie Aqua™ viability stain (Biolegend, San Diego, CA) for 20 minutes. Next, cells were washed twice with 2% FACS buffer (1×PBS and 2% BSA) centrifuged and stained with human TruStain FcX™ (Biolegend, San Diego, CA) for 20 minutes. Cells were washed twice more with 2% FACS buffer, pelleted, and stained with NKG2A (Clone S19004C, Biolegend, San Diego, CA) and anti-14G2a (Clone 1A7, National Cancer Institute, Biological Resources Branch, Rockville MD) antibodies for detection of NKG2A and CAR scFv, respectively. For phenotyping experiments, NK cells were also stained with CD56 (Clone HCD56, Biolegend, San Diego, CA), CD16 (Clone 3G8, Biolegend, San Diego, CA). Flow cytometry was performed on an Attune™ NxT flow cytometer (ThermoFisher, Waltham, MA), and data was analyzed using FlowJo.

For sorting, NK cells were either stained for NKG2A or CAR, and were sorted using a 130 m nozzle on a FACSAria™ II sorter (BD Biosciences). Sorted cells were recovered in complete NK MACS medium (Miltenyi) with 100 IU/mL IL-2 and 2 ng/mL IL-15 for four days prior to cytotoxicity assays. NK cells were assayed by flow cytometry or sorted seven days after nucleofection (Day 11). For M21 focused flow analysis, cells were trypsinized using 0.05% Trypsin (Thermo Fisher) for 5 minutes prior to filtering with a 70 m strainer, counting and plating for staining. M21 cells were stained as described above, but using an anti-GD2 antibody (Clone 14G2a, Biolegend).

LDH release cytotoxicity assay: First, 1e4 M21 cells were plated per well in a 96-well, flat bottom plate in culture medium with or without 100 ng/mL of IFNγ. 24 hours later, sorted NK cells were added to the well at the corresponding effector: target ratios in complete NK MACS medium supplemented with 100 IU/mL IL-2 and 2 ng/mL IL-15 for a total volume of 100 L. For ADCC conditions, 500 ng/mL of anti-GD2 hu14.18K322A (a gift from Paul Sondel) was added to each of the wells. After 24 hours of co-culture, the plate was centrifuged at 300 g for 5 minutes to pellet the cells. 50 μL of supernatant was harvested and assayed for secreted LDH using the CyQuant™ LDH Cytotoxicity Assay kit according to the manufacturer's instructions (ThermoFisher, Waltham, MA). The remaining supernatant was collected and frozen at −80° C. for subsequent ELISA.

IFNγ ELISA: Quantification of secreted IFNγ was carried out using an IFNγ sandwich ELISA kit (ThermoFisher) according to the manufacturer's instructions on media supernatants taken from the 24-hour LDH co-culture assay.

Time-Lapse Imaging: M21-HLA-E and NK cells were stained using DiD (ThermoFisher, Waltham, MA) and DiI (ThermoFisher, Waltham, MA) respectively. A 1:1000 dilution of each stain was prepared in PBS and cells were stained with 1 mL each for 15 minutes. M21-HLA-E cells were seeded in a 96-well flat-bottom plate at a density of 30 k cells/well 24-hours prior to starting the timelapse imaging. NK cells were added in conjunction with the apoptosis staining Caspase-3/7 Detection Reagent (ThermoFisher, Waltham, MA) at a concentration of 10 k NK cells/well. The apoptosis detection reagent was prepared by reconstituting in 100 μL PBS to prepare 100× stock solution. On the day of the experiment, a 10× working solution was prepared by diluting stock solution 1:10 in NK cell media. M21 cell media was aspirated and replaced with NK cell media containing CellEvent at a 1:1 ratio, and the plate was incubated for 15 minutes at 37° C. and 5% CO₂ immediately prior to starting the timelapse. The timelapse was conducted on a Leica Thunder Microscope with stagetop incubator at 37° C. 5% CO₂. Images were acquired every minute over a total of 16 hours. Images were processed and analyzed using FIJI. Cell killing was determined by selecting representative NK cells at the beginning of the timelapse and manually tracking their activity.

IncuCyte Assays: Ten thousand GFP-labeled CHLA-20 cells were plated per well in a 96-well, flat-bottom plate in culture medium. 24 hours later, thawed and sorted NK cell products were added to the well at 1:1 and 5:1 E:T ratios in complete NK MACS medium supplemented with 100 IU/mL IL-2 and 2 ng/mL IL-15 for a total volume of 100 L. Cell killing was measured via the IncuCyte S3 Live-Cell Analysis system, with images taken every 4 hours for 24 hours. Green object count was used to quantify the number of cancer cells per well. Cytotoxicity for a given sample was calculated as the difference between the green object count at a given time minus the count at t=0 divided by the count at t=0. Image analysis was performed using the IncuCyte Analysis Software.

In-Out PCR: Genomic DNA was extracted from 1e6 cells as described previously in the art. One primer was designed to bind upstream of the left homology arm or downstream of the right homology arm, depending on the orientation of transgene integration. The second primer was designed to bind within the scFv region of the CAR, or within the protein coding region of the mCherry transgene. PCRs were carried out using the Q5® Hot Start 2× Master Mix (NEB) according to the manufacturer's protocol for 30 cycles. On-target primer sequences are listed in Table 3 and off-target primer sequences are listed in Table 4.

TABLE 3
Primer sequences for on-target sites
On-Target

| | | | | Expected |
|---|---|---|---|---|
| | Trans- | Forward | Reverse | size |
| Locus | gene | (5′->3′) | (5′->3′) | (bp) |
| KLRC1 | CAR | TGTGCAGACCACA | Aggaaccagaa | 1349 |
| | | TAGTCTTAACCA | gccttgcagga | |
| | | SEQ ID NO: 11 | SEQ ID NO: 12 | |
| KLRC1 | mCherry | TGTGCAGACCACA | TGCCTTAGTGCCC | 1156 |
| | | TAGTCTTAACCA | TCGGACACA | |
| | | SEQ ID NO: 13 | SEQ ID NO: 14 | |

TABLE 4
Primer sequences for off-target sites

| Off | | | | | Expected |
|---|---|---|---|---|---|
| Target | Chromo- | | Forward | Reverse | size |
| Site | some | Locus | (5′->3′) | (5′->3′) | (bp) |

Off-Target (Forward orientation)

| | | | | | |
|---|---|---|---|---|---|
| 1 | 3 | RTP4 | AGATTTG | GGGGGTG | 1831 |
| | | | GGGGAAG | TCGTTTT | |
| | | | CACTGAA | GGCTGAG | |
| | | | GTC | G | |
| | | | SEQ ID | SEQ ID | |
| | | | NO: 15 | NO: 16 | |
| 2 | 15 | CDAN1 | CTGGCTT | GGGGGTG | 1632 |
| | | | TTGTGTT | TCGTTTT | |
| | | | ACCTGCT | GGCTGAG | |
| | | | TGAC | G | |
| | | | SEQ ID | SEQ ID | |
| | | | NO: 17 | NO: 18 | |
| 3 | 19 | ZNF146 | TATGCAG | GGGGGTG | 1457 |
| | | | GGAGCAG | TCGTTTT | |
| | | | GGAGGAT | GGCTGAG | |
| | | | ATAG | G | |
| | | | SEQ ID | SEQ ID | |
| | | | NO: 19 | NO: 20 | |
| 4 | 17 | RABEP1 | Tgggagg | ACAGTTG | 1611 |
| | | | ctgaggc | GTGCAGC | |
| | | | aggagaa | ATCAGCC | |
| | | | c | C | |
| | | | SEQ ID | SEQ ID | |
| | | | NO: 21 | NO: 22 | |
| 5 | 7 | ST7-OT3 | AATATTG | ACAGTTG | 1931 |
| | | | GCAGGAA | GTGCAGC | |
| | | | GGCAGGA | ATCAGCC | |
| | | | CAG | C | |
| | | | SEQ ID | SEQ ID | |
| | | | NO: 23 | NO: 24 | |
| 6 | 16 | SALL1 | CAAAGGG | GGGGGTG | 1810 |
| | | | ACCCTGC | TCGTTTT | |
| | | | CCACC | GGCTGAG | |
| | | | SEQ ID | G | |
| | | | NO: 25 | SEQ ID | |
| | | | | NO: 26 | |
| 7 | 5 | HAND1 | CACGGGC | GGGGGTG | 1738 |
| | | | AATCCCC | TCGTTTT | |
| | | | GCA | GGCTGAG | |
| | | | SEQ ID | G | |
| | | | NO: 27 | SEQ ID | |
| | | | | NO: 28 | |
| 8 | 1 | LRRIQ3 | TGGTCAT | GGGGGTG | 1776 |
| | | | GCTTGGA | TCGTTTT | |
| | | | AGACCCT | GGCTGAG | |
| | | | TG | G | |
| | | | SEQ ID | SEQ ID | |
| | | | NO: 29 | NO: 30 | |
| 9 | 1 | AKNAD1 | TCCAAGA | GGGGGTG | 1922 |
| | | | CTGCTTT | TCGTTTT | |
| | | | TCCTGTG | GGCTGAG | |
| | | | GG | G | |
| | | | SEQ ID | SEQ ID | |
| | | | NO: 31 | NO: 32 | |
| 10 | 2 | NRP2 | GCGCAGA | GGGGGTG | 1805 |
| | | | AGAGTGT | TCGTTTT | |
| | | | AAGAAGG | GGCTGAG | |
| | | | TCA | G | |
| | | | SEQ ID | SEQ ID | |
| | | | NO: 33 | NO: 34 | |
| 11 | 9 | LOC | CTCCATG | GGGGGTG | 2206 |
| | | 105375972 | GCAGCAT | TCGTTTT | |
| | | | TTCTCAT | GGCTGAG | |
| | | | TTGG | G | |
| | | | SEQ ID | SEQ ID | |
| | | | NO: 35 | NO: 36 | |
| 12 | 2 | ASB1 | Cacccca | GGGGGTG | 1677 |
| | | | tgcacac | TCGTTTT | |
| | | | acctcca | GGCTGAG | |
| | | | c | G | |
| | | | SEQ ID | SEQ ID | |
| | | | NO: 37 | NO: 38 | |

Off-Target (Reverse orientation)

| | | | | | |
|---|---|---|---|---|---|
| 1 | 3 | RTP4 | GGGGGTG | TGCCAAG | 1723 |
| | | | TCGTTTT | ACAAGTC | |
| | | | GGCTGAG | AGGTTTG | |
| | | | G | ATGA | |
| | | | SEQ ID | SEQ ID | |
| | | | NO: 39 | NO: 40 | |
| 2 | 15 | CDAN1 | GGGGGTG | GCCCCAT | 1829 |
| | | | TCGTTTT | TCTCCGT | |
| | | | GGCTGAG | ACCTGC | |
| | | | G | SEQ ID | |
| | | | SEQ ID | NO: 42 | |
| | | | NO: 41 | | |
| 3 | 19 | ZNF146 | GGGGGTG | TAGTGCT | 1759 |
| | | | TCGTTTT | TGACCCA | |
| | | | GGCTGAG | TGCAGAAC | |
| | | | G | C | |
| | | | SEQ ID | SEQ ID | |
| | | | NO: 43 | NO: 44 | |
| 4 | 17 | RABEP1 | GGGGGTG | CTGGCTG | 2011 |
| | | | TCGTTTT | AAGTGGG | |
| | | | GGCTGAG | CGTCCTT | |
| | | | G | G | |
| | | | SEQ ID | SEQ ID | |
| | | | NO: 45 | NO: 46 | |
| 5 | 7 | ST7-OT3 | GGGGGTG | GAATTCT | 1980 |
| | | | TCGTTTT | GTCTGGC | |
| | | | GGCTGAG | AGGTCCC | |
| | | | G | C | |
| | | | SEQ ID | SEQ ID | |
| | | | NO: 47 | NO: 48 | |
| 6 | 16 | SALL1 | GGGGGTG | TTCTTCC | 1540 |
| | | | TCGTTTT | TCTCTGT | |
| | | | GGCTGAG | CCCCTGA | |
| | | | G | GTC | |
| | | | SEQ ID | SEQ ID | |
| | | | NO: 49 | NO: 50 | |
| 7 | 5 | HAND1 | GGGGGTG | AATCAGG | 1696 |
| | | | TCGTTTT | GGCTACC | |
| | | | GGCTGAG | GTTGCG | |
| | | | G | SEQ ID | |
| | | | SEQ ID | NO: 52 | |
| | | | NO: 51 | | |
| 8 | 1 | LRRIQ3 | GGGGGTG | TGCAGAA | 1625 |
| | | | TCGTTTT | CCCATGA | |
| | | | GGCTGAG | CCAATCT | |
| | | | G | CTG | |
| | | | SEQ ID | SEQ ID | |
| | | | NO: 53 | NO: 54 | |
| 9 | 1 | AKNAD1 | GGGGGTG | AACAGCC | 2362 |
| | | | TCGTTTT | CATCTCA | |
| | | | GGCTGAG | AGGCACC | |
| | | | G | SEQ ID | |
| | | | SEQ ID | NO: 56 | |
| | | | NO: 55 | | |
| 10 | 2 | NRP2 | GGGGGTG | GCCTCTT | 1830 |
| | | | TCGTTTT | CCCGTTT | |
| | | | GGCTGAG | GAGGTTT | |
| | | | G | CT | |
| | | | SEQ ID | SEQ ID | |
| | | | NO: 57 | NO: 58 | |
| 11 | 9 | LOC | GGGGGTG | CCCTCCC | 1761 |
| | | 105375972 | TCGTTTT | CTCTGGG | |
| | | | GGCTGAG | ATAGAGT | |
| | | | G | TTT | |
| | | | SEQ ID | SEQ ID | |
| | | | NO: 59 | NO: 60 | |
| 12 | 2 | ASB1 | GGGGGTG | CTGACAT | 2066 |
| | | | TCGTTTT | TTCACCA | |
| | | | GGCTGAG | AGGGAGC | |
| | | | G | CA | |
| | | | SEQ ID | SEQ ID | |
| | | | NO: 61 | NO: 62 | |

On-target editing analysis via Next Generation Sequencing: Genomic DNA was extracted and indel formation was analyzed via next generation sequencing (Illumina) as previously described in the art. Briefly, edited amplicons were amplified and purified using genomic PCR and SPRI cleanup, respectively. A second PCR was performed with indexing primers to attach unique dual identifiers (Illumina®) to the amplicons. After a second SPRI cleanup, samples were combined and run on the Illumina® MiniSeq™ per the manufacturer's instructions and analyzed using the CRISPR RGEN software. Primer sequences are listed in Table 5.

TABLE 5
Primer sequences for on-target next
generation sequencing

| | SEQ | |
|---|---|---|
| | ID | |
| Identity | NO: | Sequence |
| Forward | 63 | TCCCTGACATCACACACTGC |
| (5′->3′) | | |
| Reverse | 64 | TGCCTTTAGGTTTTCGTTGC |
| (5′->3′) | | |

Off-target analysis via genome wide CHANGE-seq: The Gentra® Puregene® kit (Qiagen, Germantown, MD) was used to extract genomic DNA from unedited primary NK cells per the manufacturer's instructions. Genomic analysis was performed by CHANGE-Seq as described previously in the art. In short, genomic DNA underwent tagmentation with a custom Tn5-transposome, and was gap repaired with HiFi HotStart Uracil+ Ready Mix (Kapa Biosystems, Wilmington, MA). USER enzyme (NEB, Ipswich, MA) and T4 polynucleotide was then used to treat the gap-repaired DNA. DNA circularization was performed using T4 DNA ligase (NEB, Ipswich, MA), followed by treatment with a mixture of exonucleases to remove residual linear DNA. In vitro cleavage reactions were performed on the circularized DNA using SpCas9 protein (NEB, Ipswich, MA) and KLRC1 sgRNA. Cleaved products were incubated with proteinase K (NEB, Ipswich, MA), A-tailed, ligated with a hairpin adapter (NEB, Ipswich, MA), and treated with USER enzyme (NEB, Ipswich, MA). Products were amplified by PCR with the Kapa HiFi polymerase (Kapa Biosystems, Wilmington, MA), and libraries were quantified by qPCR (Kapa Biosystems, Wilmington, MA). Libraries were sequenced with 150 bp paired-end reads on the Illumina NextSeq2000 instrument. CHANGE-seq data analyses were performed as described previously.

Off-target analysis at nominated sites via Next Generation Sequencing: Indel frequency at off-target sites was assayed by the rhAmpSeq™ system (IDT). Genomic DNA was extracted from KLRC1-CAR NK cells. Pools of forward and reverse primers were designed using IDT's rhAmpSeq™ design tool to amplify the on-target and top 12 off-target sites detected by CHANGE-Seq. PCRs and library preparation were performed according to IDT's instructions. Pooled libraries were diluted and sequenced on the Illumina MiniSeq™ instrument with 150 bp paired-end reads. Data was analyzed using IDT's rhAmpSeq™ analysis tool.

Integration Site Analysis via Long Read Whole Genome Sequencing: Genomic DNA from KLRC1-CAR NK cells was extracted using the Gentra® Puregene® kit (Qiagen, Germantown, MD) per the manufacturer's instructions, and quantified using Qubit® 3.0 Fluorometer (ThermoFisher, Waltham, MA). Sequencing libraries were prepared using the Oxford Nanopore Technologies Ligation Sequencing Kit V14 (SQK-LSK114, Oxford, UK) according to the manufacturer's instructions with minimal modifications (namely, the gDNA was sheared using a Covaris g-tube prior to library preparation (Covaris, Woburn, MA), doubled FFPE and end-repair time, and doubled the amount of beads during clean up), and sequenced on the Oxford Nanopore Technologies PromethION instrument (Flowcell #: FLO-PRO114M) (Oxford, UK).

Whole genome sequencing reads were aligned to the scFv region of the CAR transgene; reads with alignment to the transgene were subsequently mapped to the human genome (GRCh38). Mapping was performed with minimap2 utilizing the first half of the detectIS pipeline. Reads were filtered at a mapq threshold of 30. Integration sites were identified from reads aligning to both the transgene and the human genome filtered with an overlap window threshold (distance between the transgene aligning and the human genome aligning segments/total length of read) of 0.15.

Statistical Analysis: All analyses were performed using GraphPad Prism (V10.1.0). For experiments with two groups, a two-tailed t test was used. For optimization experiments with more than two groups, a one-way ANOVA was used, with the Prism recommended post-test. For editing and cytotoxicity experiments with multiple donors, a two-way ANOVA was used, followed by the Prism recommended post-test. Post-tests are listed in the figure legend for each experiment. Error bars represent mean±SD. ns=p≥0.05, * for p≤0.05, ** for p≤0.01, *** for p≤0.001,**** for p≤0.0001.

### Example 1: CRISPR Editing of the NKG2A Receptor

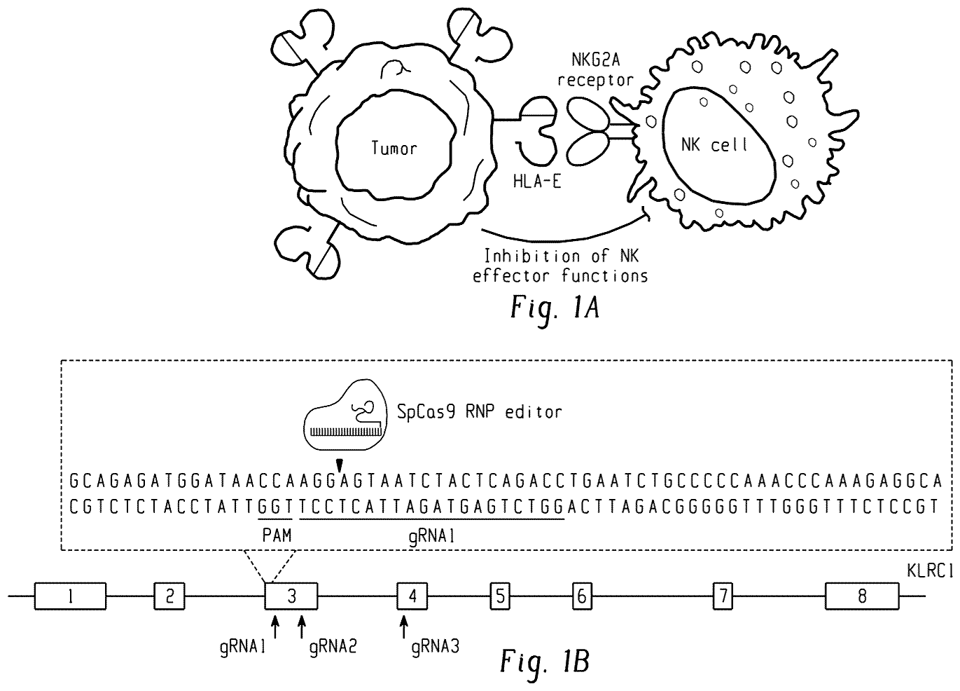

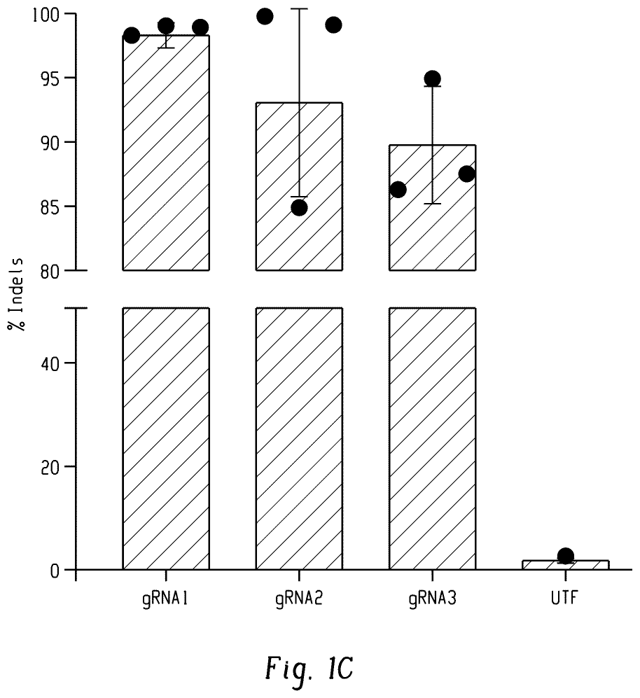

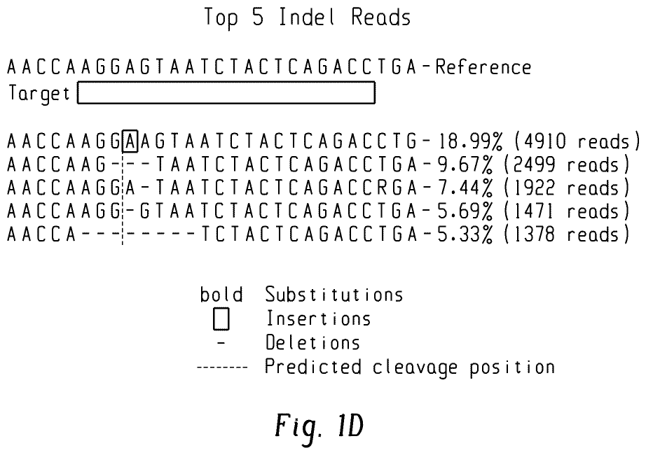

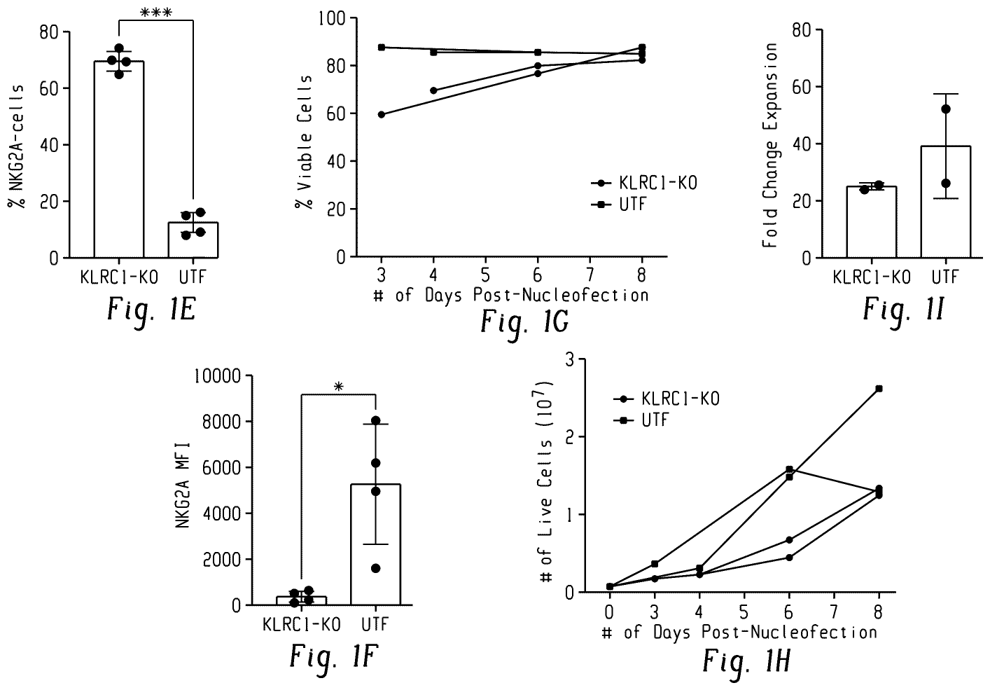

The editing strategy to improve NK potency against solid tumors involves knocking out the NKG2A receptor that can inhibit cytotoxicity within solid tumors (FIG. 1A), while simultaneously conferring specificity of cytotoxicity to a target antigen on the cancer. To knock out the NKG2A receptor, three single-guide RNAs (sgRNAs) were designed against several common coding exons in the KLRC1 gene (FIG. 1B). Ribonucleoproteins (RNPs) were generated upon the complexation of these sgRNAs with SpCas9 to generate double-stranded DNA breaks at these targets within KLRC1. NK-92 cells, a human cell line derived from non-hodgkin lymphoma, were nucleofected with RNPs containing one of these sgRNAs, and assessed for insertions or deletions (indels) around the targeted site after two weeks of cell culture. Next-generation sequencing (NGS) of PCR amplicons derived from genomic DNA showed a high level of indel formation (98%) in the RNP-treated cells for our top-performing guide RNA (gRNA1) targeting exon 3. These indels were absent in the untransfected (UTF) control sample (FIG. 1C). Moreover, the indels were centered exactly at the predicted site of DNA double-strand cleavage by SpCas9 (FIG. 1D). While NK-92 cells are highly cytotoxic and can be used as an allogenic cell therapy, their cancerous origin requires them to be irradiated before infusion, therefore limiting their in vivo persistence. Additionally, their lack of CD16 expression renders them incapable of antibody dependent cellular cytotoxicity (ADCC). For these reasons, KLRC1^{-/-} cells were generated, termed KLRC1-KO, using primary NK cells isolated from peripheral blood.

NK cells were isolated from the whole blood of healthy donors using density-based negative selection and activated by co-culture with irradiated K562-mb15-41BBL feeder cells supplemented with IL-2 and IL-15. After four days of culture, NK cells were nucleofected with an RNP complex targeting the KLRC1 gene and expanded for one week. The edited cells were analyzed for NKG2A expression using flow cytometry alongside a donor-matched UTF control population. Within the RNP-treated cells, an average of 70% were negative for NKG2A on their cell surface, in stark contrast to the 13% within the UTF control (FIG. 1E). The loss of surface NKG2A protein is further corroborated by the decrease in the mean fluorescence intensity (MFI) signal in the RNP-treated cells when compared to the UTF cells (FIG. 1F). To ensure that knockout of NKG2A did not affect cell growth, cell viability and proliferation of the KLRC1-KO cells were tracked after nucleofection. KLRC1-KO cells maintained high viability throughout expansion and proliferated consistently (FIGS. 1G, 1H). Depending on the donor, the fold expansion of KLRC1-KO cells was similar or marginally lower than that of their UTF counterparts (FIG. 1I).

### Example 2: Non-Viral Transgene Knock-In into KLCR1

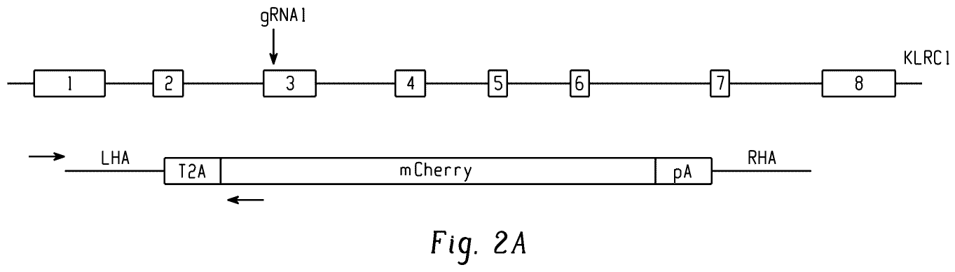

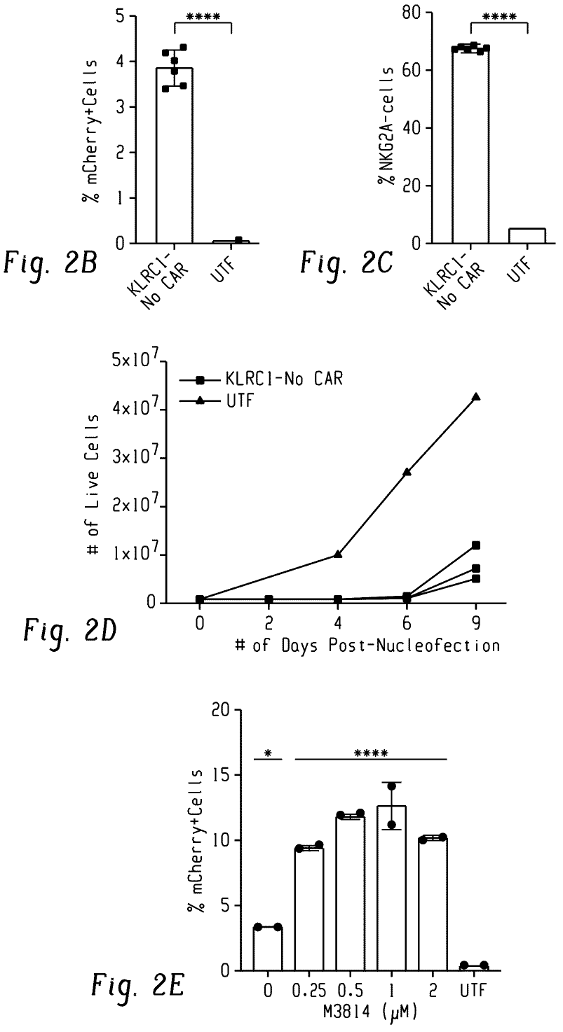

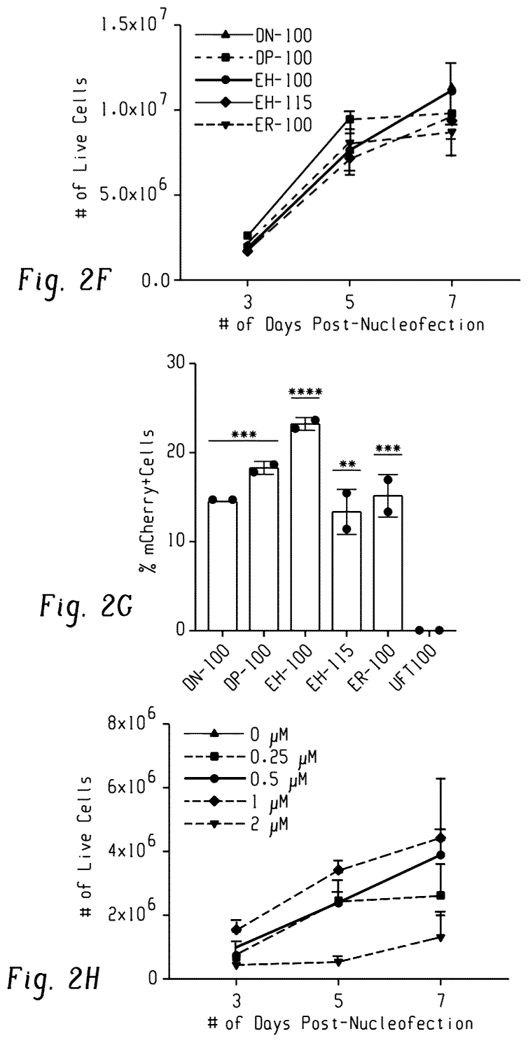

To assess whether this editing strategy could be used to insert long transgenes in primary NK cells, a virus-free editing technique developed for primary T cells that harnesses homology-directed repair (HDR) at double-strand DNA breaks was adapted. NK cells were nucleofected with an RNP complex together with a linear, double stranded DNA (dsDNA) template encoding a long transgene (2709 bp): the signaling-inert mCherry gene (KLRC1-No CAR) (FIG. 2A). Since HDR is most likely to occur when cells are actively proliferating and entering the S- or G2-phases of the cell cycle, the optimal time to nucleofect the cells was determined to ensure the highest knock-in efficiencies. NK cells were nucleofected on days 4-7 of expansion and the level of NKG2A knockout and mCherry expression were assessed by flow cytometry. mCherry integration was highest when cells were nucleofected on day 4 with an average of 4% cells exhibiting mCherry expression and 68% cells demonstrating NKG2A knockout (FIGS. 2B, 2C). However, very low cell growth of these cells was observed for the first six days after nucleofection (FIG. 2D). On days 5, 6, and 7, the percentage of mCherry integration significantly decreased to only about 1% (data not shown). In contrast, the percentage of NKG2A⁻ cells increased to 80% when cells were nucleofected on days 5, 6, and 7 respectively (data not shown).

To increase HDR, the DNA-dependent protein kinase (DNA-PK) inhibitor, M3814, was used as it has been shown to improve knock-in rates in primary human T cells by inhibiting NHEJ events. NK cells were nucleofected on day 4 with the RNP complex and dsDNA, and incubated with M3814 at various concentrations. Nucleofected cells were expanded for one week and assayed by flow cytometry for NKG2A knock-out and transgene expression. Incubation with M3814 had no effect on NKG2A knock-out efficiencies, with the percent of NKG2A⁻ cells remaining around 75% (data not shown). In contrast, knock-in efficiencies significantly improved when nucleofected cells were treated with any concentration of M3814 compared to no M3814 treatment (FIG. 2E). Cells treated with either 0.5 μM or 1 μM of M3814 had the highest percentage of transgene expression at 11.9% and 12.8%, respectively. Moreover, treatment with M3814 did not affect cell expansion after nucleofection, with M3814-treated cells growing comparably to untreated cells (FIG. 2F). For subsequent studies, the 0.5 μM concentration was chosen due to the increased editing efficiency and high cell yield at one week after nucleofection.

Since this editing strategy is electroporation-based, the electrical pulse used to deliver the gene editing cargo is highly important. For the Lonza 4D nucleofection system, two programs were selected (DN-100 and EH-100) that have resulted in high RNP based knock-out efficiencies and plasmid-based GFP knock-in efficiencies while maintaining cell viability in primary NK cells. Additionally, two pulse codes were included (DP-100 and ER-100) that were recommended by the manufacturer as being similar to DN-100 but potentially yielding higher efficiencies. As a control, the EH-115 program used to nucleofect primary human T cells was tested. Consistent with the M3814 optimization, there was no significant difference in NKG2A knock-out efficiencies across the different programs, with the percent of NKG2A⁻ cells remaining between 76-85% (data not shown). The EH-100 program yielded the highest percentage of transgene-positive cells at 23.5% (FIG. 2G). Finally, cell viability and growth throughout expansion was assessed for each of the pulse code programs to maximize cell yield. Throughout expansion, the DN-100 program resulted in higher cell yields, while the ER-100 program yielded the lowest number of viable cells. (FIG. 2H) The EH-100 pulse code was chosen for subsequent experiments due to the significant improvement in transgene editing and sufficient expansion of the edited cells.

### Example 3: Characterization of KLRC1-CAR NK Cells

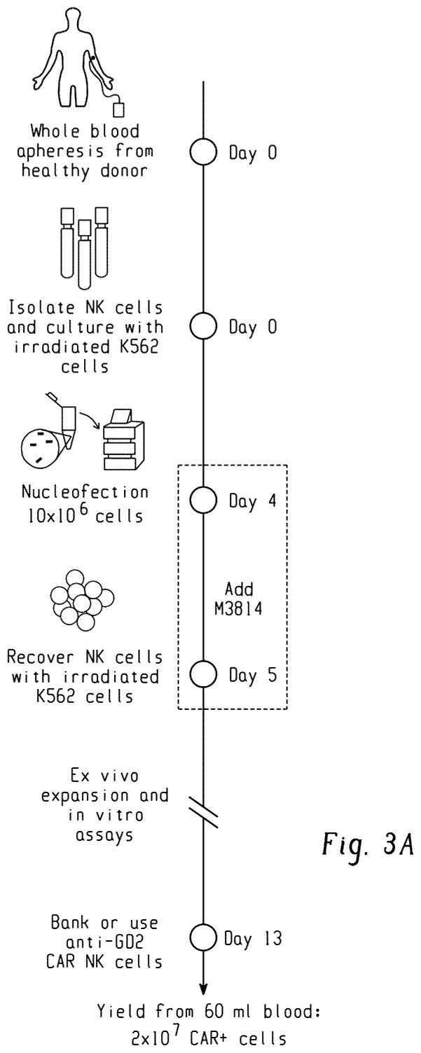

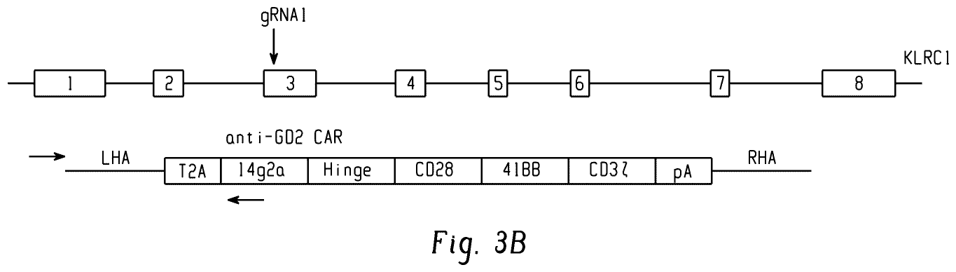

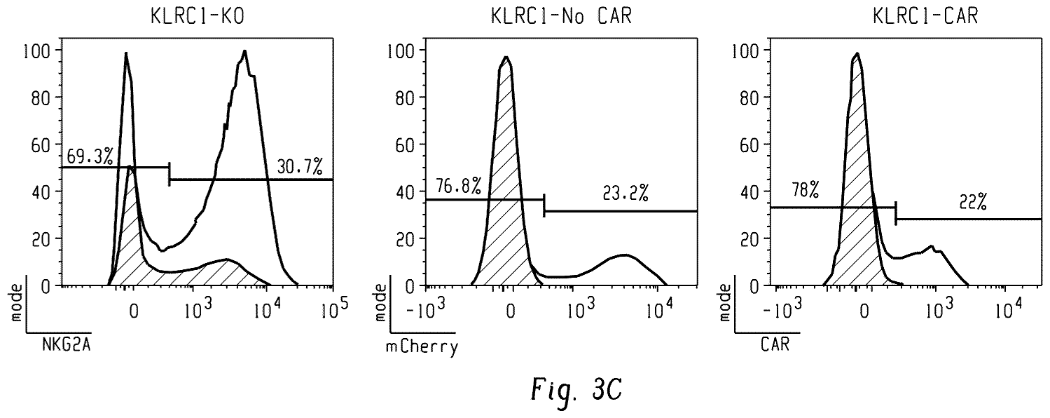

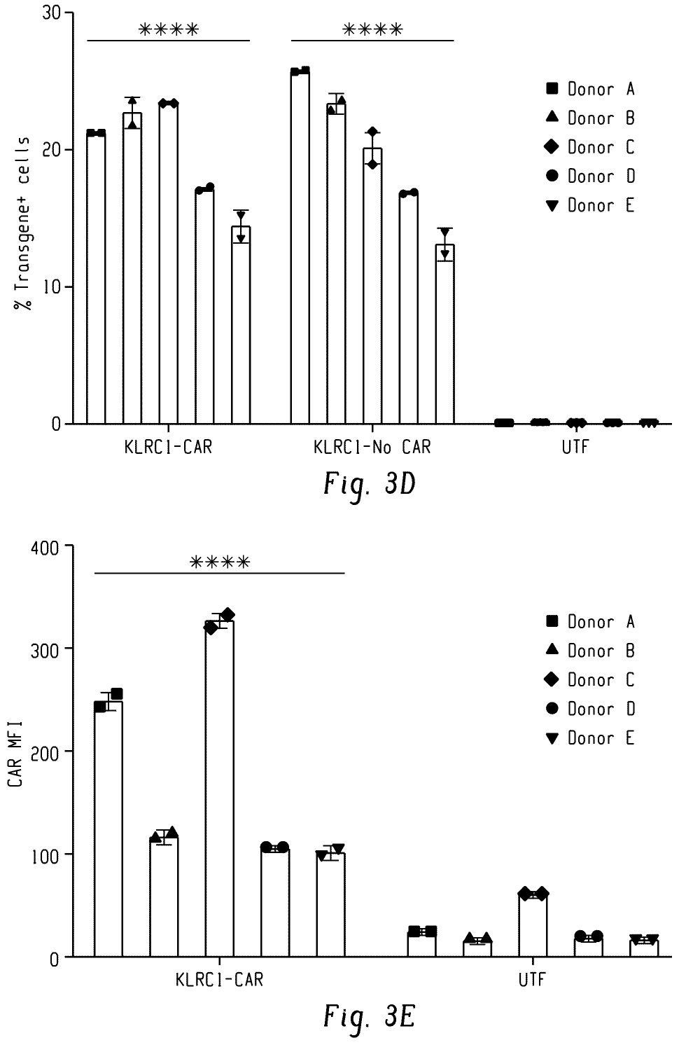

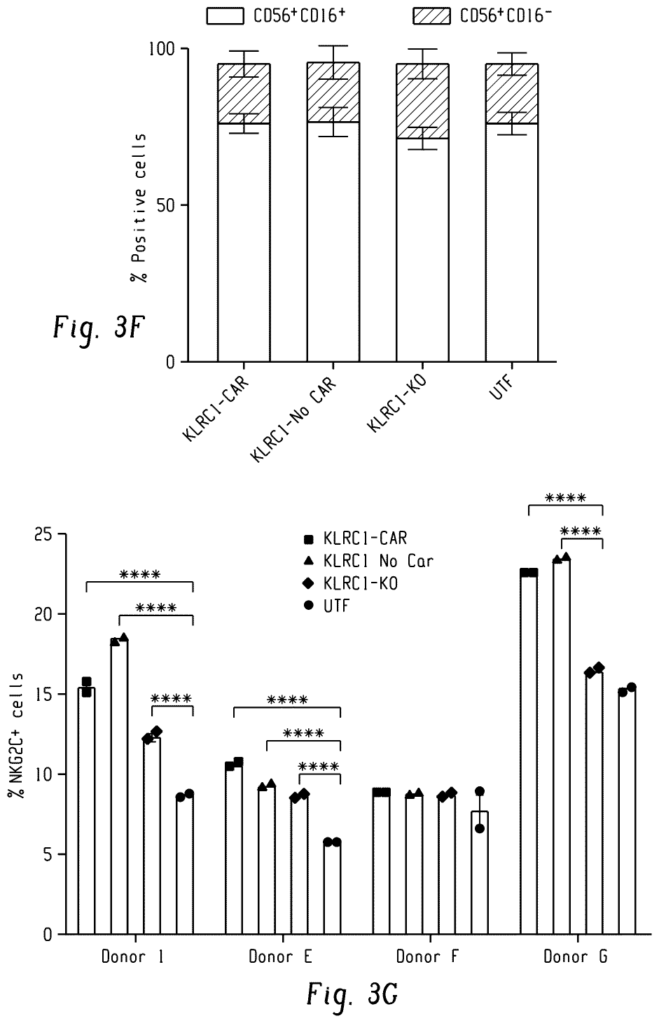

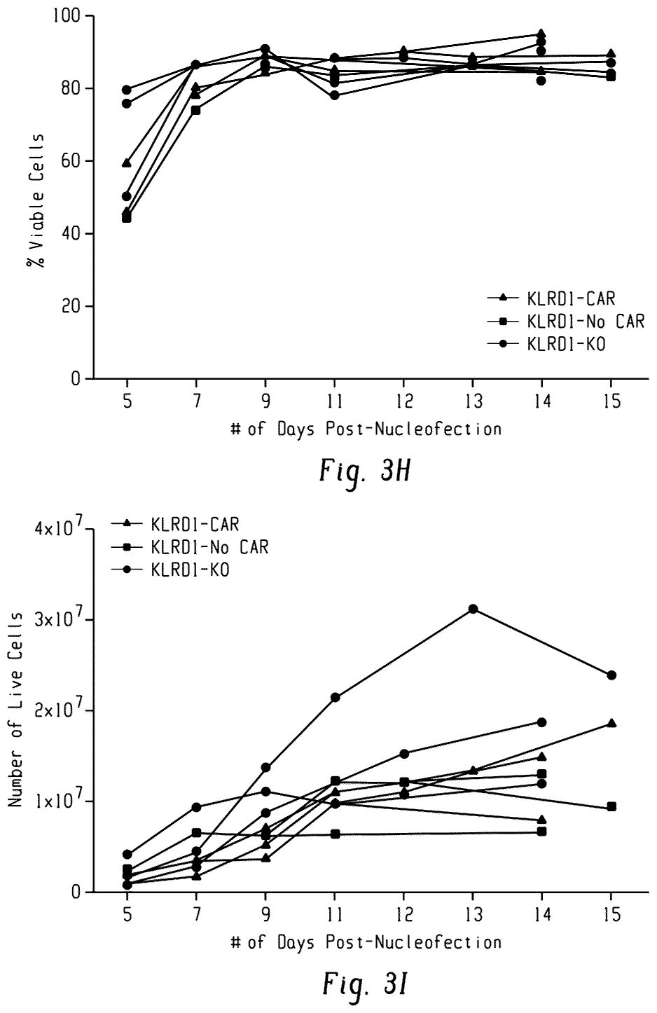

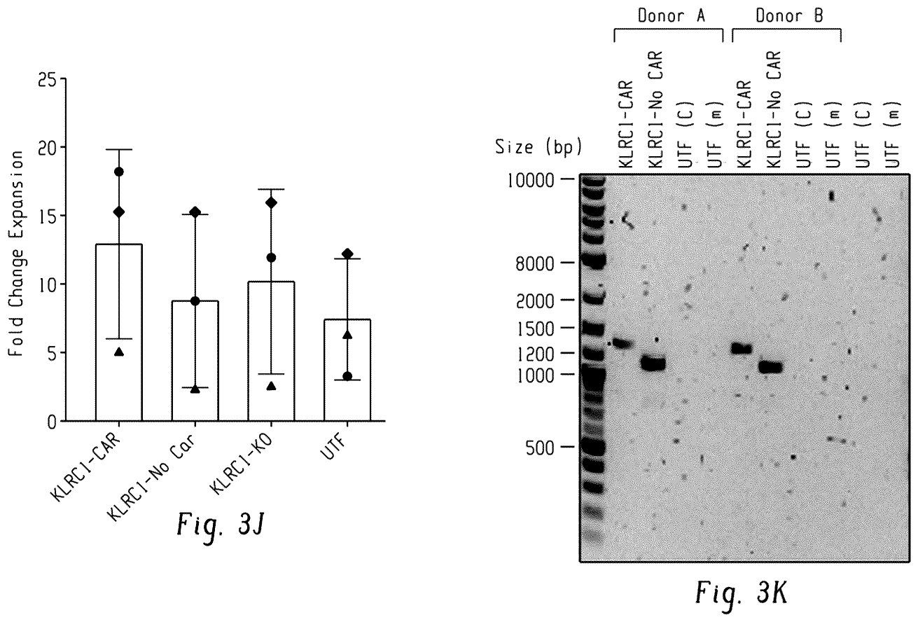

Having developed an efficient nonviral KLRC1 gene editing protocol for NK cells (FIG. 3A), the strategy was applied to generate GD2-targeting CAR NK cells (KLRC1-CAR), with an anti-GD2 CAR composed of the following: an anti-GD2 scFv (14g2a); CD28 transmembrane and endodomain; 41BB and CD3z cytotoxicity domains (FIG. 3B). Though the CAR transgene is approximately 500 bp larger than the mCherry transgene, the CAR knock-in efficiency (20.0%) is similar to that of the mCherry template (20.1%) (FIGS. 3C, 3D). The MFI for the CAR transgene is also significantly increased compared to the donor-matched UTF control (FIG. 3E). In line with previous outcomes, the NKG2A knock-out efficiencies remained largely unchanged when cells received the CAR transgene but were increased marginally with the delivery of the mCherry transgene (data not shown). The NKG2A MFI levels, however, had no significant differences across the three groups that were edited via CRISPR (data not shown). After profiling for canonical CD56 and CD16 NK cell surface markers in the cell products, the fractions of CD56⁺CD16⁺ and CD56⁺CD16⁻ cells among the edited and untransfected groups remained unchanged through the editing and expansion workflows (FIG. 3F). In contrast, a donor-dependent increase in NKG2C expression was observed among the edited groups in comparison to UTF controls, with KLRC1-CAR and KLRC1-No CAR cells harboring the greatest expression (FIG. 3G).

We next confirmed that insertion of the CAR into the KLRC1 locus did not affect NK cell viability and proliferation by tracking cell expansion after nucleofection. When comparing the various cell products immediately after nucleofection, poor cell expansion correlated with the length of the exogenous dsDNA template within the first week, with KLRC1-CAR NK cells having the lowest fold expansion, followed by KLRC1-No CAR, and KLRC1-KO NK cells (data not shown). However, after recovery from electroporation, KLRC1-CAR NK cells were highly viable and proliferated at a similar rate to KLRC1-No CAR and KLRC1-KO NK cells in the log growth phase (FIGS. 3H, 3I). The fold change expansion for all edited groups beginning on day 5 was comparable to that of donor matched UTF NK cells with no significant differences (FIG. 3J).

The genomic outcomes within the KLRC1-CAR NK cell product were characterized by checking for on- and off-target modifications, and integration of our transgenes throughout the genome. First, an “in-out” PCR for the intended transgene integration at the KLRC1 on-target site was performed on genomic DNA extracted from edited NK cells. Using primers that were specific to the transgene and the KLRC1 locus, we showed site-specific amplification of our GD2 CAR and mCherry transgenes, that were not present in the UTF cells (FIG. 3K). The size of the bands are approximately 1300 bases (CAR) and 1100 bases (mCherry), exactly as predicted from the proper on-target integration of each transgene.

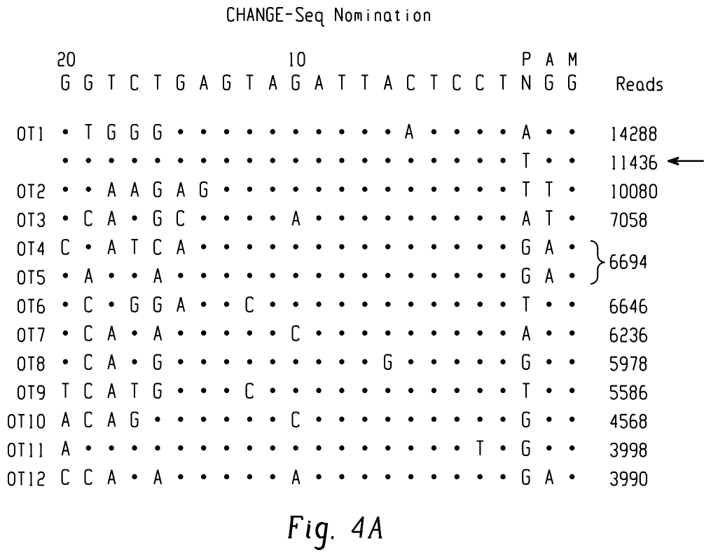

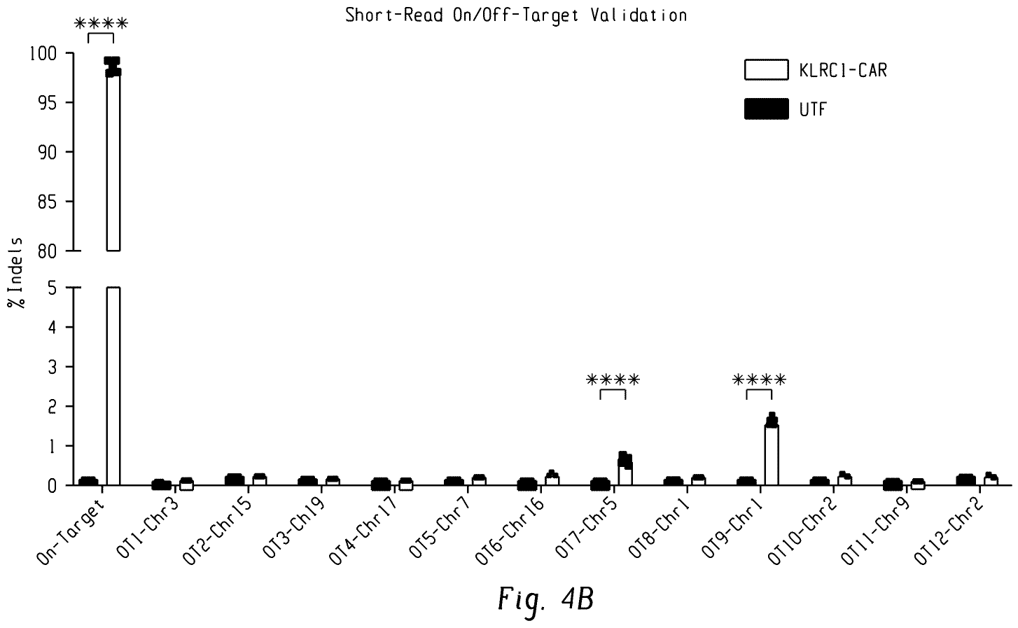

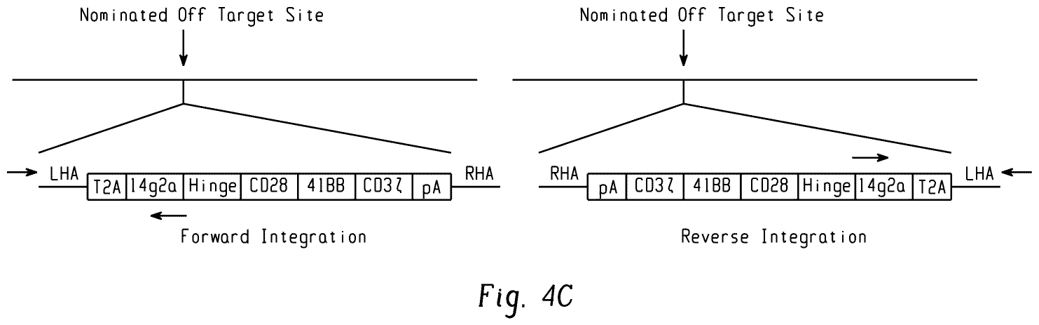

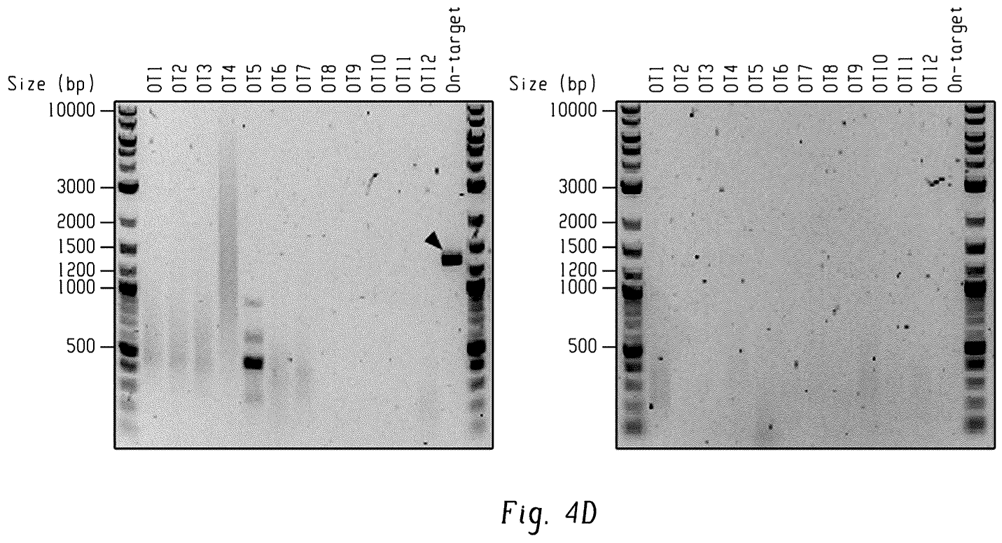

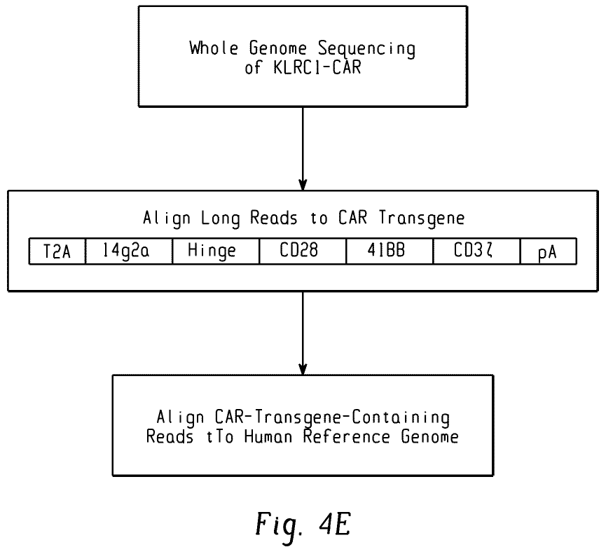

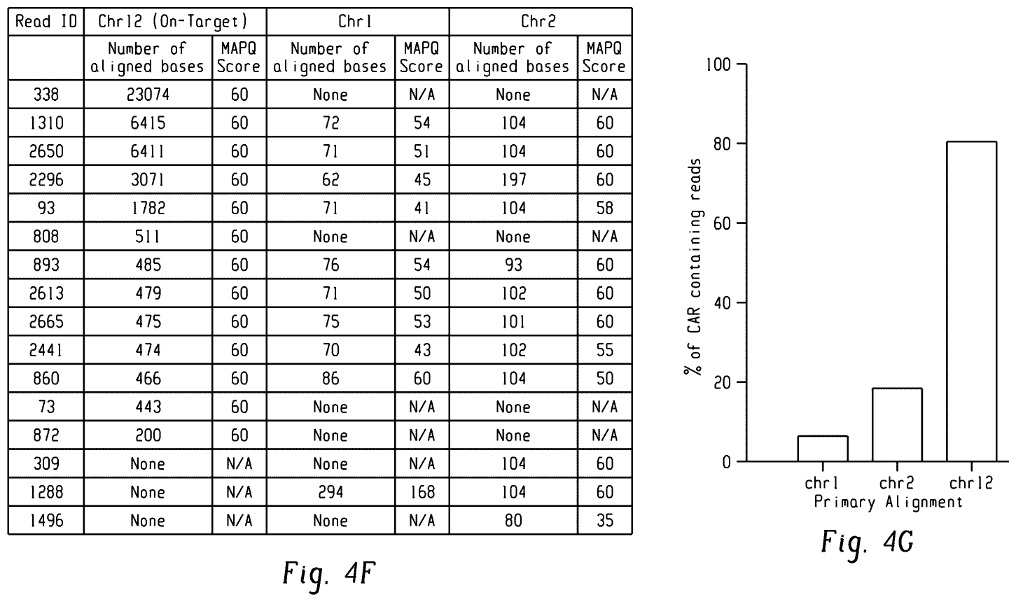

We also examined the genome for off-target activity from the KLRC1 gRNA via CHANGE-seq. This method profiles the entire human genome for double-stranded DNA breaks induced by a Cas9 RNP. Moderate off-target activity in the CHANGE-seq assay was observed largely within intergenic or intron regions of the genome, with 10 sites having off-target nuclease activity within exons and 8 sites having off-target activity near a transcription start site (TSS) (data not shown). The CHANGE-seq specificity ratio, calculated by dividing the number of on-target reads by the sum of total reads, for our sgRNA is 0.038. As expected, small mismatches distal to the protospacer-adjacent motif (PAM) of Cas9 were present in the top nominated off-target sites (FIG. 4A).

To further evaluate potential off-target genomic modifications, amplicon sequencing was performed around each of the top 12 off-target sites nominated by CHANGE-seq (>3500 reads) within KLRC1-CAR and UTF NK cells. NGS was completed via the rhAmpSeq analysis system to multiplex the genomic analysis for on- and off-target sites from the same genomic DNA sample. By rhAmpSeq, high indel formation was observed only at the on-target site with low or undetectable levels of editing across all 12 off-target sites (FIG. 4B). At two sites, modest but significant increase in off-target activity was observed compared to UTF controls.

To evaluate the potential off-target integration of the CAR, in-out PCR was performed on each of the top 12 off-target sites across three donors to check for unintended CAR integration, in either the 5′-3′ or 3′-5′ orientation (FIG. 4C). No bands of the expected size were observed in any of the off-target sites, indicating that there is no off-target integration of the CAR within the limit of detection for this assay (FIG. 4D). The band at ˜500 bp at site 5 (FIG. 4D, left, lane 6) is from non-specific binding of the primers as the expected amplicon size from off-target integration at this site would have been 1931 bp. To confirm that the band observed at ˜500 bp was from non-specific binding of the primers, PCR analysis of site 5 was repeated alongside donor-matched UTF controls. Amplification of the ˜500 bp band was observed within both KLRC1-CAR and UTF groups (data not shown), confirming non-specific primer activity. As a positive control, the on-target site was included, and a band of approximately 1300 bp was observed (FIG. 4D), which corresponds to the expected amplicon size of 1349 bp, confirming successful on-target integration of the CAR within KLRC1.

Long read whole genome sequencing (WGS) of KLRC1-CAR NK cells was performed to track integration sites of the transgene unbiasedly, as WGS does not have amplification biases from PCR nor does it require nomination of off-target sites. Genomic DNA was prepared for long-read sequencing via Oxford Nanopore Technologies (ONT) and aligned to a reference sequence containing the CAR transgene (FIG. 4E). Successful mapping to the reference human genome provided 30× coverage of the genome. On-target integration was observed in 13 of 16 reads with alignment to the CAR transgene. An exact match to the predicted homology-directed repair product at the intended on-target KLRC1 locus on Chromosome 12 was observable in several distinct long reads spanning the insertion and homology arms (FIGS. 4F and 4G). These single reads had the highest possible score (MAPQ of 60) and showed contiguous mapping to Chromosome 12 as far as 7000 bases upstream and 4000 bases downstream of the arms (FIGS. 4F, 4G). Of the reads containing the CAR transgene, 81% of them mapped to portions of the on-target site with high scores within the specified overlap window (FIG. 4G). The remaining 19% of the reads mapped partially to the CAR and chromosomes 1 and 2. However, the alignment length of these reads is quite small compared to the on-target alignments: on average, only 71 bp alignment for Chromosome 1, and 101 bp for Chromosome 2 versus 1362 bp for Chromosome 12 (FIG. 4F). Upon closer examination, the alignment of the reads on Chromosomes 1 and 2 mapped to CAR domains that are present in the native human genome, such as CD28 on Chromosome 1 and CD3-zeta on Chromosome 2. Overall, WGS results confirmed high levels of precise on-target integration from the genome editing strategy described herein.

### Example 4: KLRC1-CAR Cells are Highly Cytotoxic Against Cells with the Target Antigen

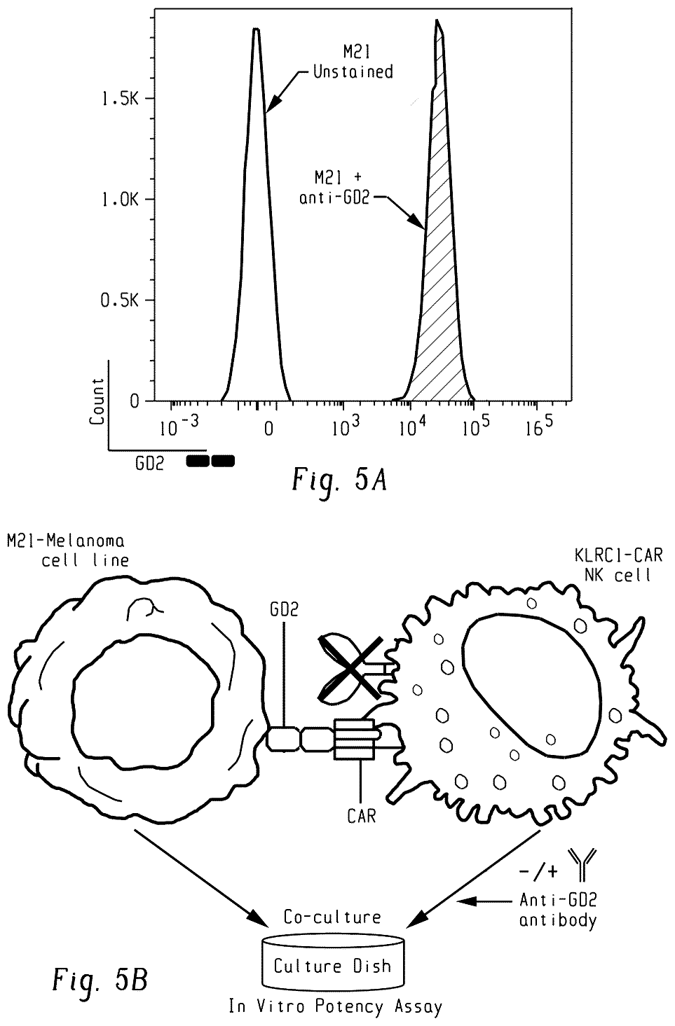

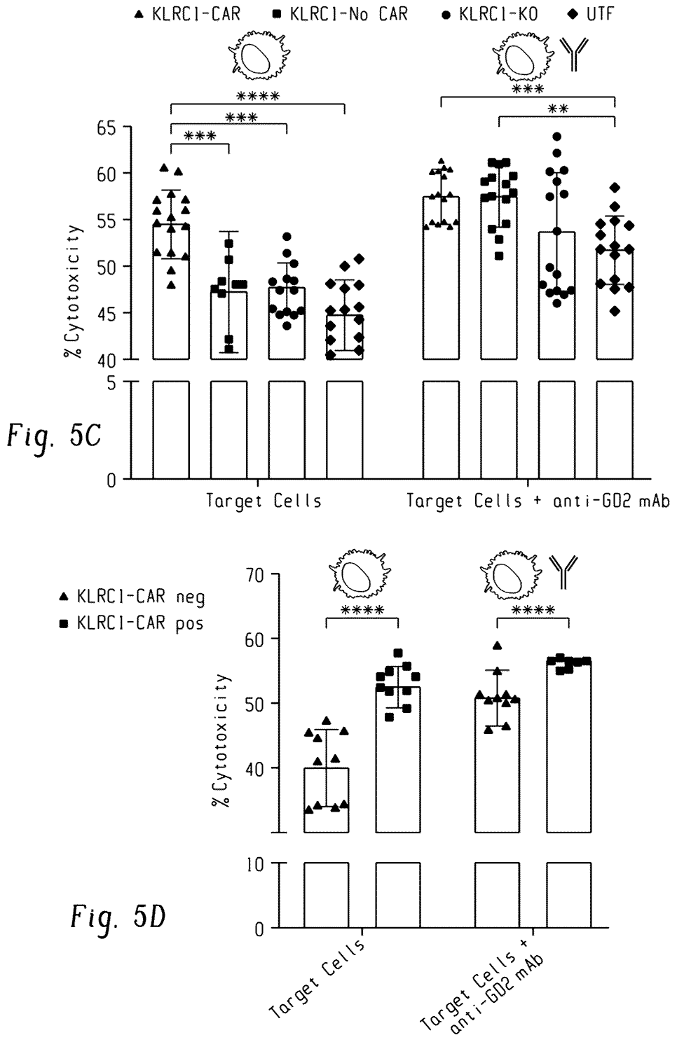

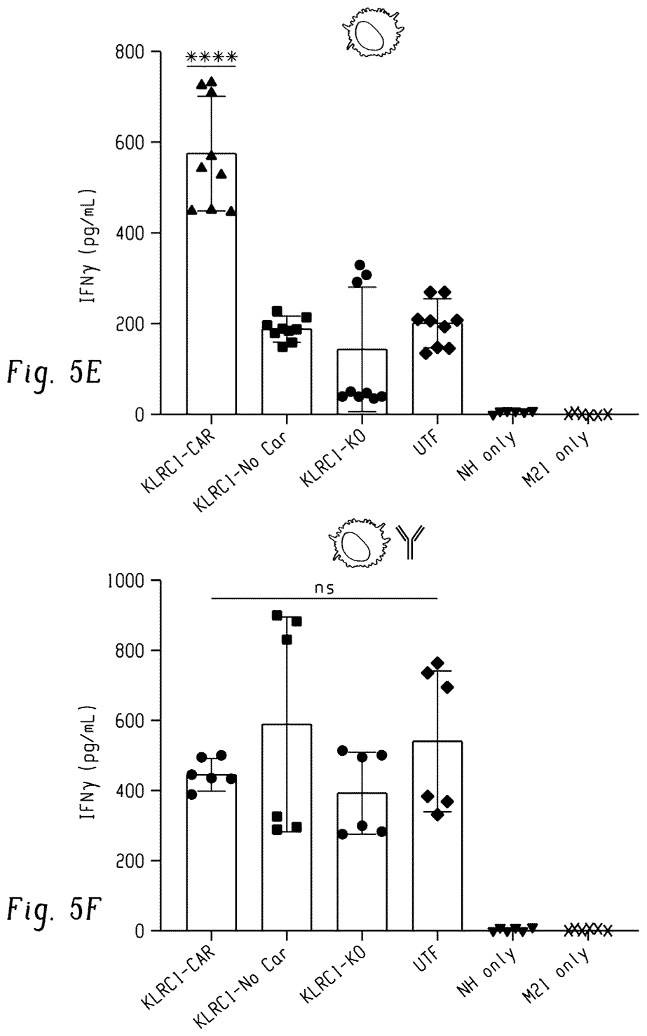

The specificity of KLRC1-CAR NK cell function was evaluated against GD2⁺ M21 melanoma cells (FIG. 5A). Pure populations of NKG2A⁻, CAR⁺, or mCherry⁺ cells were isolated using florescence activated cell sorting (FACS) to determine the effect of each gene modification on NK cytotoxicity. NK cells were co-cultured with M21 cells at a 5:1 effector-target ratio for 24 hours (FIG. 5B). NK cytotoxicity was 6-10% higher in the KLRC1-CAR NK cells compared to KLRC1-No CAR, KLRC1-KO and UTF cells (FIG. 5C, left). A monoclonal antibody to label the CAR target antigen, GD2, was added to the co-culture so that NK cell recognition of the tumor could occur independent of the CAR via CD16, triggering ADCC. Upon addition of the hu14.18K322A anti-GD2 monoclonal antibody, all NK cell products were highly cytotoxic (FIG. 5C, right) and the differences in NK cell cytotoxicity between KLRC1-CAR, KLRC1-No CAR, KLRC1-KO NK cells were erased in ADCC promoting conditions. Upon comparing non-ADCC and ADCC conditions, we attribute that the boost in the cytotoxic activity of the KLRC1-CAR NK cells compared to other cells is specific to the GD2 antigen. The precise knock-in of the CAR increases the cytotoxicity of the KLRC1-CAR NK cells to the maximal level in this co-culture assay.

To assess whether the improvement in cytotoxicity within the KLRC1-CAR NK cells was observed across other tumor models, the engineered NK cell products were cultured with GFP-labeled, GD2-expressing CHLA-20 neuroblastoma cells and measured NK cell killing. At the 1:1 E:T, KLRC1-CAR⁺ NK cells displayed the highest cytotoxicity compared to KLRC1-CAR⁻ and KLRC1-No CAR at the 8-, 12-, and 24-hour time points (data not shown). As the number of NK cells was increased to reach an E:T of 5:1, the differences between KLRC1-CAR cells and other NK cell populations were minimized within the 24-hour window.

To further verify that the increase in cytotoxicity is due to the CAR, CAR⁺ and CAR⁻ cell fractions were isolated within a sample and NK cytotoxicity was measured. The CAR⁺ fractions exhibited 13% greater cytotoxicity than their CAR⁻ counterparts (FIG. 5D). Again, this difference between the two populations was completely erased upon addition of the anti-GD2 monoclonal antibody under ADCC conditions.

Analysis of media supernatant indicated that the KLRC1-CAR NK cells secreted the greatest amount of IFNγ upon antigen exposure (FIG. 5E). In agreement with the cytotoxicity data, there were no significant differences in IFNγ production between the KLRC1-No CAR, KLRC1-KO, and UTF groups. Under ADCC conditions, all four groups produced an equivalent amount of IFNγ after antigen-exposure (FIG. 5F). Interestingly, all groups except the KLRC1-CAR NK cells secreted a greater total amount of IFNγ when compared to their non-ADCC counterparts.

### Example 5: KLRC1-CAR Cells Overcome HLA-E Mediated NK Cell Inhibition

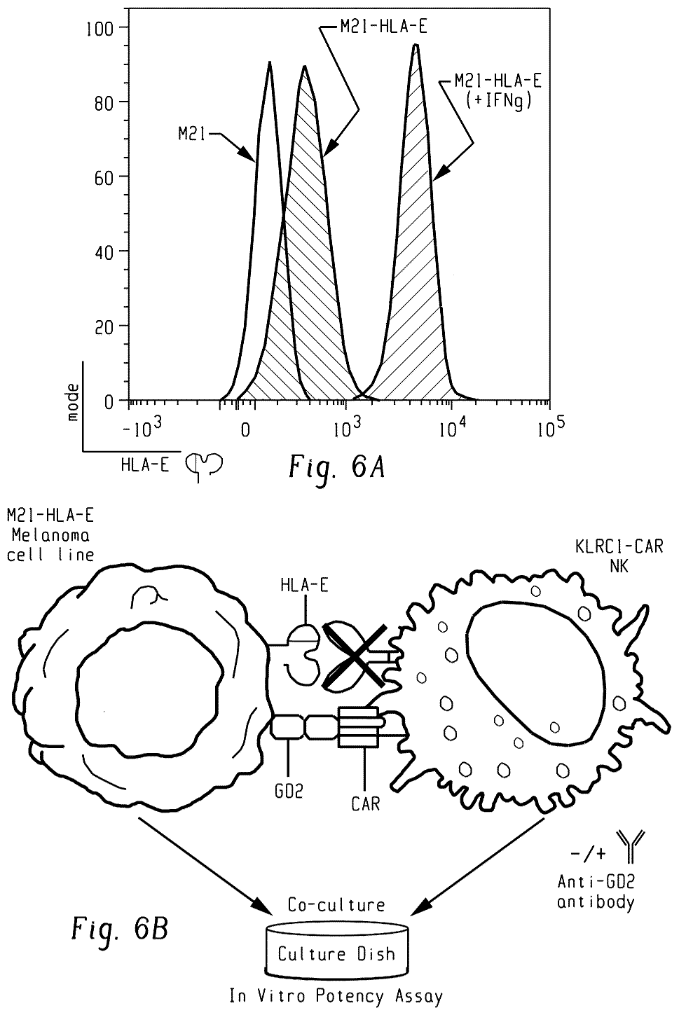

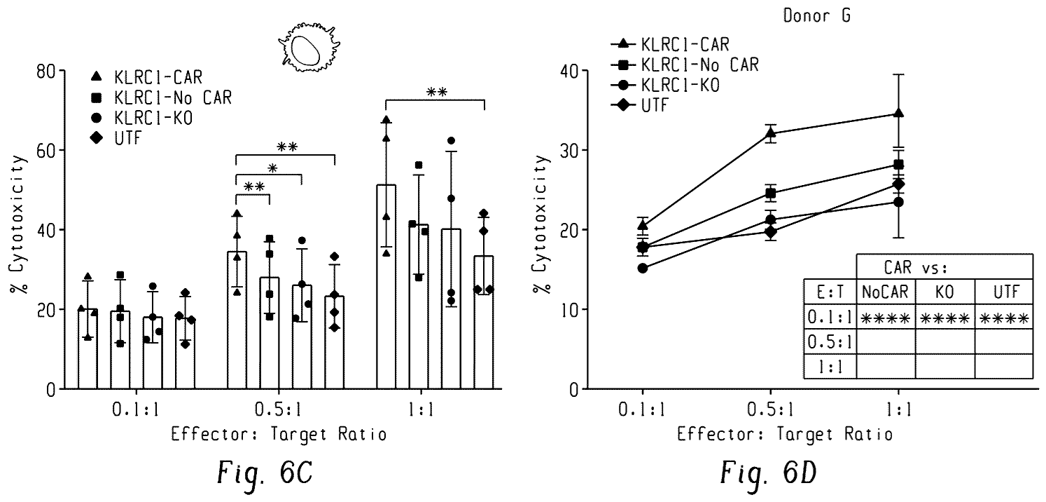

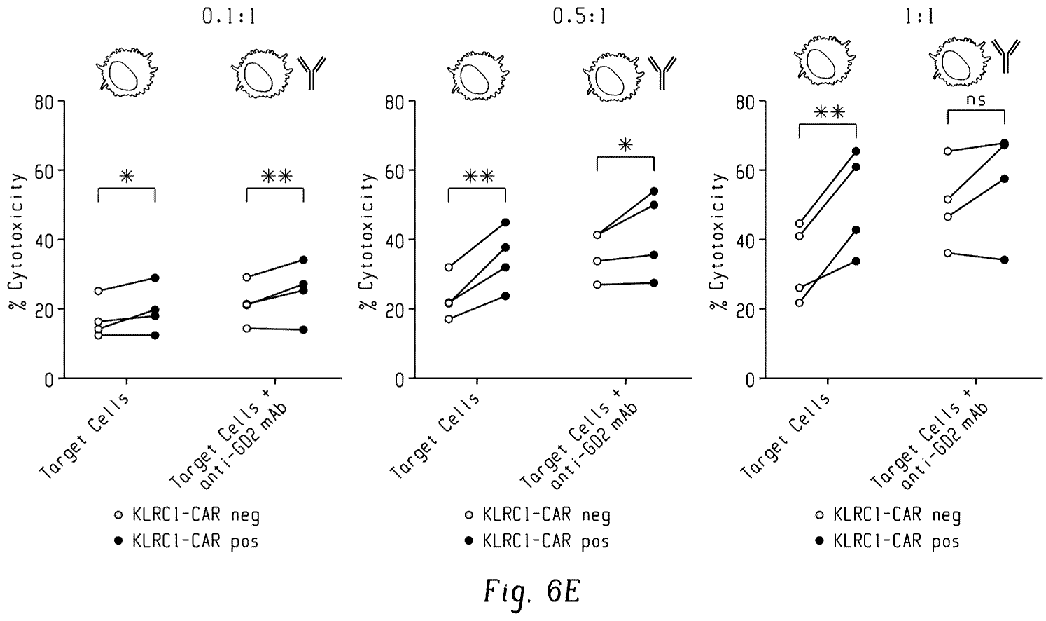

To interrogate the combinatorial effect of the NKG2A knock-out and CAR insertion, the potency of the KLRC1-CAR NK cells against HLA-E expressing M21 cells was assessed. Following transduction with an HLA-E vector containing an HLA-G leader peptide, 44% of M21 cells expressed HLA-E on the cell surface, with expression increasing to 100% after a 24-hour treatment with 100 ng/mL of IFNγ (FIG. 6A). Pure populations of NKG2A⁻, CAR⁺, or mCherry⁺ cells were isolated using FACS and co-cultured with IFNγ-treated M21-HLA-E cells at various effector-target ratios for 24 hours alongside donor-matched UTF controls (FIGS. 6B-D, and data not shown). At the lowest ratio (0.1:1), there were no differences in NK-mediated cytotoxicity between the groups. When cultured at the 0.5:1 ratio, KLRC1-CAR NK cells displayed superior cytotoxicity compared to KLRC1-No CAR, KLRC1-KO, and UTF cells. At the highest ratio, KLRC1-CAR NK cells continued to exhibit improved cytotoxicity when compared to UTF cells by 17% respectively, but no significant differences were observed relative to the KLRC1-No CAR or KLRC1-KO NK cells. Again, in ADCC conditions, the differences in cytotoxicity were nullified between the edited groups at all three effector-target ratios, mirroring the trends seen against the wild-type M21 cells (data not shown). Finally, it was confirmed that the improvement in NK potency against HLA-E expressing cancer cells was still driven by CAR activity by analyzing the cytotoxicity of CAR⁺ and CAR⁻ fractions from within the same sample (FIG. 6E). The CAR⁺ fractions displayed improved cytotoxicity at each of the effector: target ratios by 2%, 11%, and 18% respectively. Under ADCC conditions, these differences were minimized at the 0.5:1 and 1:1 ratios to 5% and 7% respectively, with a slight increase at the 0.1:1 ratio to 3.5%.

### Example 5: KLRC1-CAR NK Cells Exhibit Greater Killing Per Cell

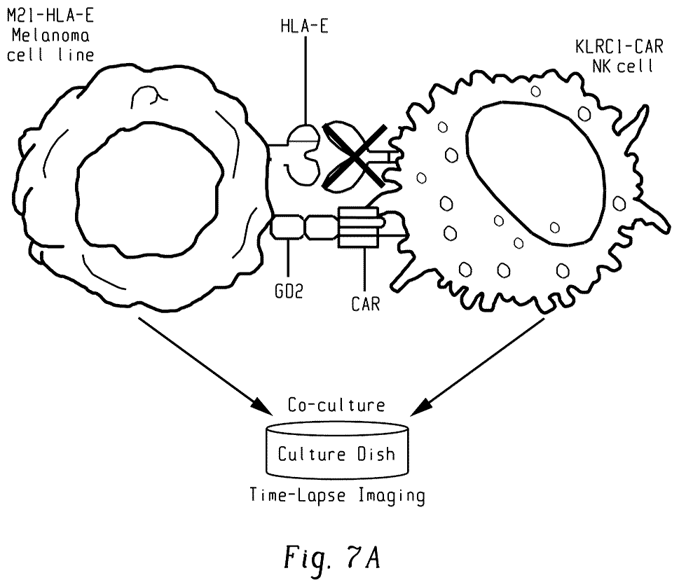

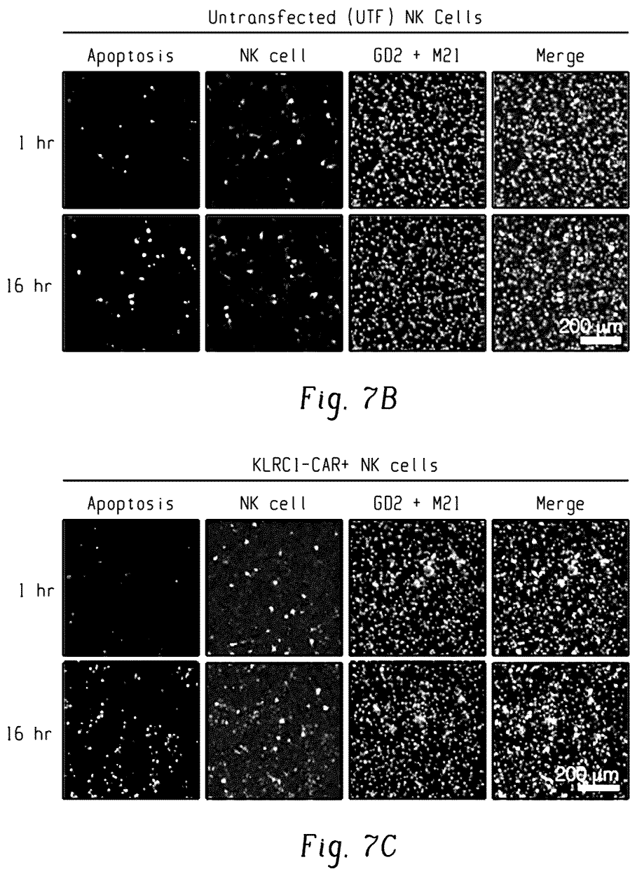

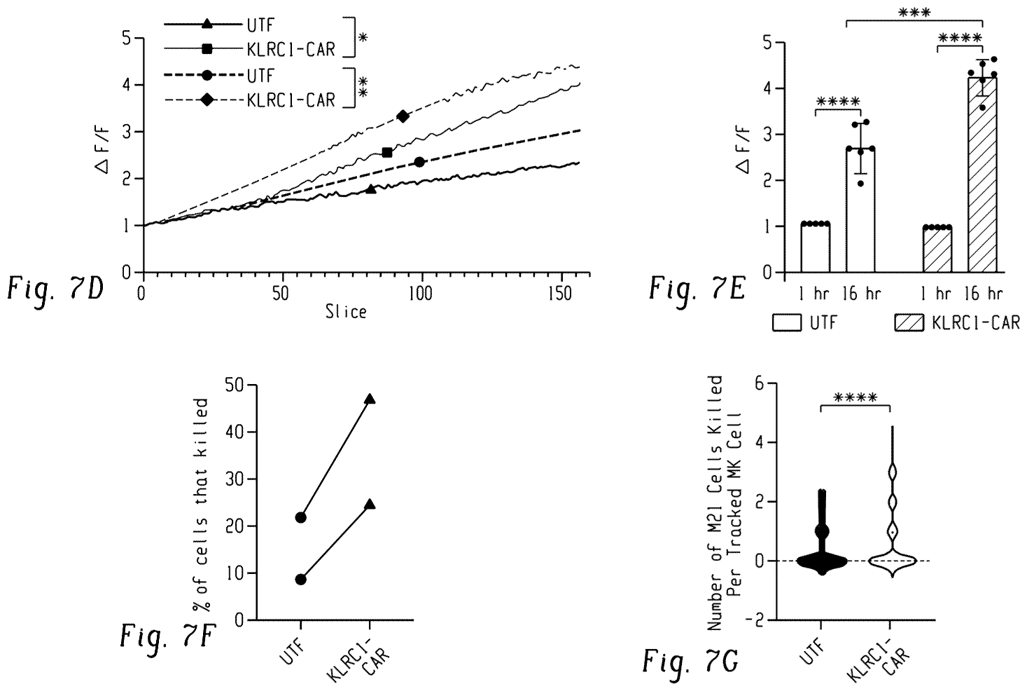

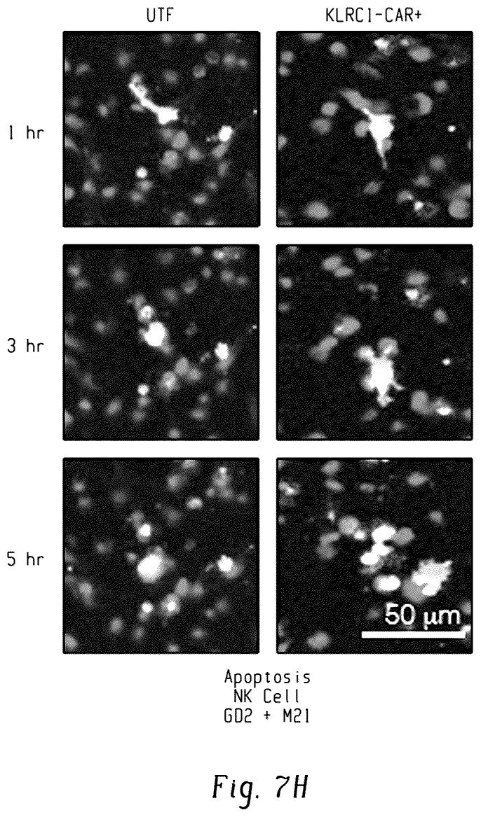

To track KLRC1-CAR NK cell cytotoxicity at a single-cell resolution, the dynamics between the NK cells and the M21-HLA-E cells were imaged through timelapse imaging (FIG. 7A). KLRC1-CAR NK or UTF NK cells were co-cultured with M21-HLA-E cells and were imaged for 16 hours (FIGS. 7B and 7C). An increase in apoptotic fluorescence intensity was observed over the course of the co-culture within both effector groups, with the KLRC1-CAR NK cells inducing a faster rate of increase, suggesting a quicker and more potent response (FIG. 7D). When comparing the final apoptotic fluorescence intensity values for each effector group at 1 and 16 hours, KLRC1-CAR NK cells displayed a greater increase in signal intensity than their UTF counterparts (FIG. 7E). For analysis at the single-cell level, 15 representative NK cells were chosen per condition and tracked for the number of M21-HLA-E cells they killed. A higher percentage of KLRC1-CAR NK cells displayed cytotoxic capacity against the target cells when compared to donor-matched UTF controls (FIG. 7F). Moreover, KLRC1-CAR NK cells also demonstrated the ability to kill multiple M21-HLA-E cells simultaneously, unlike UTF controls, suggesting that the improvement in potency could partly be attributed to “burst killing” (FIGS. 7G and 7H). Importantly, the KLRC1-CAR NK cells display better target killing per NK cell tracked, indicating the genome editing strategy produces potent functional responses against HLA-E expressing target cells at the single cell level.

### DISCUSSION

NK cells often fail to control solid tumors due to the immunosuppressive conditions of the tumor microenvironment, which can include the expression of ligands (e.g., HLA-E) that engage inhibitory receptors on NK cells like NKG2A. Though NK cells have previously been genetically modified to successfully improve cytotoxicity and persistence using viral vectors, transposons, mRNA transfection, and CRISPR+AAV technologies, these methods elicit concerns of safety, lack the machinery for stable editing, or have knock-in limitations on transgene size. Herein, an optimized CRISPR-Cas9-enabled approach that generates stable and precisely edited primary NK cells without the use of viral vectors is described. The data show that the CRISPR platform can be leveraged for efficient knock-out of the inhibitory NKG2A receptor and virus-free knock-in of an anti-GD2 CAR. A high knock-in efficiency within primary NK cells is demonstrated for large transgenes (˜3.1 kb) with little to no off-target editing. Moreover, it was validated that KLRC1-CAR NK cells are viable, proliferative and capable of inducing tumor lysis and cytokine secretion in vitro. Taken together, this work establishes that ex vivo expanded NK cells from peripheral blood can be effectively modified to express a functional CAR, while also deleting an immune checkpoint molecule using CRISPR genome editing.

Despite nominating many off-target sites for the sgRNA from the highly sensitive CHANGE-seq assay, minimal indel formation was observed across the off-target sites. One explanation is that the RNP complex is present in the NK cells transiently, resulting in lower exposure to nuclease activity when compared to the in vitro assay conditions used in CHANGE-Seq. Furthermore, M3814 may be inhibiting NHEJ events in cells that experienced an off-target double-stranded break, resulting in either cell death or precise HDR. Notably, these findings are similar to that of the recently-approved exagamglogene autotemcel (exa-cel, Casgevy™) Cas9-edited cell therapy, in which none of the nominated sites were seen upon validation in the cell product. The negligible indel formations at sites 7 and 9 within the KLRC1-CAR NK cells were found to be in the HAND1 and AKNAD1 genes, respectively, neither of which are expressed by NK cells (Human Protein Atlas: Immune Cell Section Summary). While the gRNA utilized in this study has a promising on/off target profile, it is possible to decrease the off-target activity further by choosing other guides or utilizing nucleases other than SpCas9. Importantly, little to no off-target integration was observed using the assays described in this study, indicating that the endogenous KLRC1 promoter is likely the sole driver of CAR expression of our promoter-less CAR transgene. Though virally engineered CAR NK cells have not been associated with NK cell lymphomas in patients, the CRISPR strategy described in this study increases the safety profile of NK cell therapies by lowering random transgene integrations, thus establishing a more precise and controlled therapeutic.

The RNP based knock-out strategy described herein yields high numbers of KLRC1^{-/-} cells that are viable and proliferative, at efficiencies higher than those previously reported (Bexte, T. et al. CRISPR-Cas9 based gene editing of the immune checkpoint NKG2A enhances NK cell mediated cytotoxicity against multiple myeloma. Oncoimmunology 11, 2081415 (2022); Mac Donald, A. et al. KLRC1 knockout overcomes HLA-E-mediated inhibition and improves NK cell antitumor activity against solid tumors. Front. Immunol. 14, 1231916 (2023)). Bexte et al. reported a knock-out efficiency of 43.5%, while Mac Donald et al. reported an efficiency of 41% for sgRNAs targeting the third exon. Interestingly, Bexte et al. reported a lower indel frequency for the sgRNA used in this study (˜58-80%) in primary T cells. However, the protein expression data was not reported in that study, which makes it difficult to conclude how the editing translates to knock-out efficiencies in their system. In contrast, Huang et al. (Huang, R. S., Lai, M. C., Shih, H. A. & Lin, S. A robust platform for expansion and genome editing of primary human natural killer cells. J. Exp. Med. 218, (2021)), reported high indel frequencies (>80%) for the sgRNA used in this study, but showed less than 25% NKG2A knock-out. Though Donald et al. delivers a greater amount of nuclease, their system uses the Cas12a enzyme, highlighting a difference between the studies.

The dsDNA template strategy used in this study for transgene integration has been utilized in primary NK cells before, but for smaller sequences. The studies from Huang et al. (Huang, R. S., Lai, M. C., Shih, H. A. & Lin, S. A robust platform for expansion and genome editing of primary human natural killer cells. J. Exp. Med. 218, (2021)) and Shy et al. (Shy, B. R. et al. High-yield genome engineering in primary cells using a hybrid ssDNA repair template and small-molecule cocktails. Nat. Biotechnol. 41, 521-531 (2023)) report knock-in efficiencies ranging from 3.09%-7.5% for a dsDNA GFP template (˜1.5 kb) across several loci. One study by Kath et. al (Kath, J. et al. Integration of ζ-deficient CARs into the CD3-zeta gene conveys potent cytotoxicity in T and NK cells. bioRxiv 2023.11.10.565518 (2023) doi:10.1101/2023.11.10.565518) report knock-in outcomes of less than 10% for a truncated CAR of approximately 1.4 kb. Despite using transgenes that are larger than those published previously, the methods described herein achieved similar knock-in rates (without HDR enhancers), suggesting that templates in particular size ranges (i.e., 1.5 kb to 3 kb) may produce similar editing outcomes. Since HDR is more likely to occur within cells in the S- or -G2 phases, the best knock-in percentages were observed on day 4 when the NK cells were activated with K562-mb15-41BBL feeder cells, IL-2, and IL-15 for the first four days. As the K562 cells are eliminated from the culture beyond day 4, the NK cells may enter a less activated state even with cytokine stimulation, which would explain the gradual decrease in editing on days 5-7. Two major differences between the system described herein and the previous experiments is the length and type of activation prior to nucleofection. Regardless of whether cytokines, CD2/CD335 coated beads, or K562 cells are used to activate the NK cells, for two or four days, comparable knock-in rates were observed across all three systems. Collectively, these findings suggest that activated NK cells are most amenable to genome engineering 2-4 days after isolation/thaw, with day 4 being a more optimal time point if feeder cells are employed to ensure a pure NK population.

Using an NHEJ inhibitor and fine-tuning of the pulse code improved knock-in outcomes. The DNA-PK inhibitor, M3814, improved HDR outcomes by two-fold in the system described herein. Interestingly, these results contrast with that of previous studies where other HDR enhancers and DNA-PK inhibitors had no effect on editing efficiencies within NK cells. Despite being in the same class of drugs, M3814 and KU00060648 elicit a different effect on cell viability and editing. An increase in editing is observed by using a stronger pulse code (EH-100) that likely increases the permeabilization of the cells, allowing for better cargo delivery without affecting cell viability. Surprisingly, the EH-115 program resulted in the lowest percent of mCherry⁺ cells at 13.8%, which is in contrast of the previous results. This discrepancy may be due to the fact that this system uses a linear, dsDNA template for transgene insertion instead of a plasmid. The DN-100 program resulted in knock-in efficiencies of 15%, which is approximately double what was published in Huang et al. for the same dsDNA template strategy.

NK cells are challenging to genetically engineer due to their poor recovery post-editing. Their innate lineage equips them with pattern recognition receptors (PRR) and Toll-Like receptors (TLR) that can recognize cytosolic viral and dsDNA molecules, thus increasing dsDNA associated toxicity. In agreement with this, slower proliferation of the KLRC1-CAR NK and KLRC1-No CAR cells was observed in the first five days following electroporation and a lower overall yield after expansion, compared to the KLRC1-KO and UTF cells. Without being held to theory, it is hypothesized that the co-delivery of a dsDNA template along with the RNP complex is toxic to the cells and might be causing activation of cGAS-STING, AIM2, or Type I-IFN signaling, ultimately suppressing cell proliferation and inducing apoptosis. Given that the templates for PCR for dsDNA template production are transformed and grown in E. coli, and purified in-house, it is also possible that the templates contain trace amounts of endotoxins that may further exacerbate the dsDNA associated toxicity.

Described herein is compelling evidence that the CRISPR platform can be utilized to generate functional CAR NK cells using a primary cell source. When exposed to both wild-type and HLA-E expressing M21 cells, KLRC1-CAR NK cells display greater target lysis and IFNγ secretion compared to all other groups. In the HLA-E expressing model, the difference between KLRC1-CAR and UTF NK cells is further widened even when cultured at a lower effector-target ratio (9% for 5:1 E:T versus 27% for 1:1 E:T), underscoring the importance of disrupting the NKG2A-HLA-E checkpoint axis. Our data also suggests that NKG2A knock-out and CAR expression have an additive effect on NK potency against solid tumors. However, in ADCC conditions, we observe no differences in killing between the KLRC1-CAR, KLRC1-No CAR, and KLRC1-KO cells, highlighting the impact of CAR engagement and signaling in improving NK cytotoxicity. The increase in cytotoxicity of all groups except the KLRC1-CAR NK cells in ADCC conditions also indicates that the CAR NK cells already display maximal cytotoxic capacity on their own, while the KLRC1-No CAR, KLRC1-KO, and UTF NK cells require the addition of a monoclonal antibody to achieve comparable activity.

Altogether, demonstrated herein is the feasibility of using the CRISPR system to genetically modify primary NK cells, and of driving CAR expression and activity via the KLRC1 locus. This CRISPR based platform can enable virus-free manufacturing of an off-the-shelf, genetically engineered NK cell therapy.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A genetically modified natural killer (NK) cell, comprising a knock-out of at least a portion of an endogenous gene encoding an NKG2A receptor which inactivates the NKG2A receptor, and a knock-in of a transgene for a chimeric antigen receptor (CAR) into the site of the NKG2A knock-out, wherein the CAR comprises an extracellular domain linked to an intracellular domain through a first transmembrane domain, wherein the first extracellular domain comprises an antigen recognition domain.

2. The genetically modified NK cell of claim 1, wherein the NK cells are allogenic NK cells.

3. The genetically modified NK cell of claim 1, wherein the antigen recognition domain binds an autoimmune or inflammatory disease antigen, a cardiac disease antigen, a senescence disease associated antigen, a disease associated antigen, or a tumor-specific antigen.

4. The genetically modified NK cell of claim 3, wherein the tumor-specific antigen comprises carbonic anhydrase IX (CAIX), carcinoembryonic antigen (CEA), CD5, CD8, CD7, CD10, CD19, CD20, CD22, CD30, CD33, CLL1, CD34, CD38, CD41, CD44, CD49f, CD56, CD74, CD133, CD138, CD123, CD44V6, Claudin-18, B7 homolog 3 protein (B7-H3), fibroblast activation protein (FAP), cancer antigen 19 (CA19), an antigen of a cytomegalovirus (CMV) infected cell, epithelial glycoprotein-2 (EGP-2), epithelial glycoprotein-40 (EGP-40), epithelial cell adhesion molecule (EpCAM), receptor tyrosine-protein kinases erb-B2,3,4 (erb-B2,3,4), folate-binding protein (FBP), fetal acetylcholine receptor (AChR), adult AChR subunits, folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Ra2), κ-light chain, kinase insert domain receptor (KDR), Lewis Y (LeY), L1 cell adhesion molecule (LlCAM), melanoma antigen family A, 1 (MAGE-A1), Mucin 16 (MUC16), Mucin 1 (MUC1), Mesothelin (MSLN), PSMA, GPC3, ERBB2, MAGEA3, p53, MART1, GP100, Proteinase3 (PR1), Tyrosinase, Survivin, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGFR, EGFR-vIII, CD99, CD70, ADGRE2, CCR1, LILRB2, PRAME CCR4, CD5, CD3, TRBC1, TRBC2, TIM-3, Integrin B7, ICAM-1, CD70, Tim3, CLEC12A, ERBB, or a combination thereof.

5. The genetically modified NK cell of claim 3, wherein the tumor-specific antigen comprises a tumor antigen selected from GD2, HER2, EGFR, mesothelin, Claudin-18.2, PSMA, B7-H3, IL-13Ra2, FAP, CA19, CD19, CD5, MUC1, or a combination thereof.

6. The genetically modified NK cell of claim 1, wherein the transmembrane domain comprises CD4, CD8, CD-8 alpha, CD8-beta, CD3-epsilon, CD3-beta, CD28, 4-1BB, OC40, PD-1, LAG-3, CH2CH3 or NKG2D, IgG, CD3-ζ, or a combination thereof, or wherein the single transmembrane domain comprises CD4, CD8, CD-8 alpha, CD8-beta, CD3-epsilon, CD3-beta, CD28, 4-1BB, OC40, PD-1, LAG-3, CH2CH3 or NKG2D, IgG, CD3-ζ, or a combination thereof, and/or wherein the intracellular domain comprises a costimulatory domain selected from 2B4, CD27, CD28, CD137, CD154, CD244, CD278, and combinations thereof, and a signaling domain selected from CD16, DAP10, DAP12, CD28, ICOS, CD27, OX40, CD40L, CD3-ζ, and combinations thereof.

7. An ex vivo, non-viral method of site-specifically inserting a transgene containing a chimeric antigen receptor (CAR) gene into an endogenous NK cell gene, to provide genetically modified NK cells, wherein the endogenous NK cell gene encodes an NKG2A receptor, the method comprising
providing a non-viral homology-directed repair (HDR) template comprising the transgene flanked by homology arms that are complementary to sequences on both sides of a cleavage site in the endogenous NK cell gene, wherein the CAR comprises an extracellular domain linked to an intracellular domain through a first transmembrane domain, wherein the first extracellular domain comprises an antigen recognition domain that binds a tumor-specific antigen, and wherein cleavage at the cleavage site inactivates the NKG2A receptor; and
introducing into a population of unmodified NK cells a Cas9 ribonucleoprotein (RNP) complex and the double-stranded HDR template, to provide the genetically modified NK cells
wherein the Cas9 RNP complex comprises a Cas9 protein and a guide RNA that directs double stranded DNA cleavage of the cleavage site in the endogenous NK gene, and
wherein, after introducing the Cas9 RNP and the HDR template, the endogenous NK cell gene is knocked-out and the transgene is specifically knocked-in to the knock-out site, to provide the genetically modified NK cells.

8. The method of claim 7, wherein introducing the Cas9 ribonucleoprotein (RNP) and the HDR template into the population of unmodified NK cells is done in the presence of a DNA-dependent protein kinase (DNA-PK) inhibitor.

9. The method of claim 7, wherein the population of unmodified NK cells is expanded for 4-7 days prior to introducing the Cas9 ribonucleoprotein (RNP) and the double-stranded HDR template.

10. The method of claim 9, wherein expansion is done in the presence of feeder cells.

11. The method of claim 7, further comprising expanding the genetically modified NK cells in to provide an expanded population of genetically modified NK cells.

12. The method of claim 7, wherein in the genetically modified NK cells, an endogenous promoter of the endogenous NK cell gene drives expression of the transgene, or wherein the transgene includes a promoter that drives expression of the transgene.

13. The method of claim 7, wherein the homology arms have a length of 400 to 1000 base pairs.

14. The method of claim 7, wherein the non-viral double-stranded HDR template sequentially comprises a left homology arm-a self-cleaving peptide sequence-CAR gene-a polyA terminator-a right homology arm.

15. The method of claim 14, wherein the self-cleaving peptide sequence is a T2A coding sequence.

16. The method of claim 7, wherein the NK cells are allogenic NK cells.

17. The method of claim 7, wherein the tumor-specific antigen comprises carbonic anhydrase IX (CAIX), carcinoembryonic antigen (CEA), CD5, CD8, CD7, CD10, CD19, CD20, CD22, CD30, CD33, CLL1, CD34, CD38, CD41, CD44, CD49f, CD56, CD74, CD133, CD138, CD123, CD44V6, Claudin-18, B7 homolog 3 protein (B7-H3), fibroblast activation protein (FAP), cancer antigen 19 (CA19), an antigen of a cytomegalovirus (CMV) infected cell, epithelial glycoprotein-2 (EGP-2), epithelial glycoprotein-40 (EGP-40), epithelial cell adhesion molecule (EpCAM), receptor tyrosine-protein kinases erb-B2,3,4 (erb-B2,3,4), folate-binding protein (FBP), fetal acetylcholine receptor (AChR), adult AChR subunits, folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Ra2), κ-light chain, kinase insert domain receptor (KDR), Lewis Y (LeY), L1 cell adhesion molecule (LlCAM), melanoma antigen family A, 1 (MAGE-A1), Mucin 16 (MUC16), Mucin 1 (MUC1), Mesothelin (MSLN), PSMA, GPC3, ERBB2, MAGEA3, p53, MART1, GP100, Proteinase3 (PR1), Tyrosinase, Survivin, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGFR, EGFR-vIII, CD99, CD70, ADGRE2, CCR1, LILRB2, PRAME CCR4, CD5, CD3, TRBC1, TRBC2, TIM-3, Integrin B7, ICAM-1, CD70, Tim3, CLEC12A, ERBB, or a combination thereof.

18. The method of claim 7, wherein the transmembrane domain comprises CD4, CD8, CD-8 alpha, CD8-beta, CD3-epsilon, CD3-beta, CD28, 4-1BB, OC40, PD-1, LAG-3, CH2CH3 or NKG2D, IgG, CD3-ζ, or a combination thereof, or wherein the single transmembrane domain comprises CD4, CD8, CD-8 alpha, CD8-beta, CD3-epsilon, CD3-beta, CD28, 4-1BB, OC40, PD-1, LAG-3, CH2CH3 or NKG2D, IgG, CD3-ζ, or a combination thereof, and/or wherein the intracellular domain comprises a costimulatory domain selected from 2B4, CD27, CD28, CD137, CD154, CD244, CD278, or a combination thereof, and a signaling domain selected from CD16, DAP10, DAP12, CD28, ICOS, CD27, OX40, CD40L, CD3-ζ, and combinations thereof.

19. A method of treating an individual in need of NK cell therapy by administering to the subject the genetically modified NK cells of claim 1.

20. The method of claim 19, wherein the individual is in need of treatment for a solid tumor.

21. The method of claim 20, wherein the solid tumor comprises a sarcoma, adrenocortical carcinoma, retinoblastoma, kidney cancer, bladder cancer, breast cancer, neuroblastoma, melanoma, sarcoma, neuroendocrine cancer, colorectal cancer, lung cancer, head and neck cancer, prostate cancer, pancreatic cancer, ovarian cancer, uterine cancer, oral cavity cancer, glioblastoma, lymphoma, diffuse midline glioma, carcinoid tumors, neuroendocrine tumors, thyroid cancer, liver cancer, or a combination thereof.
